SEQUENCE LISTING (57 sequences)

SEQ ID NO: 1 - DNA, 85 bp, Escherichia coli
ttgaaaatgcaatggttgactcaggtaaaatgtatggctcccataaattatttaatacatcagttcctggtttgtattacacatt

SEQ ID NO: 2 - DNA, 609 bp, Escherichia coli
accacgtgacttattaaagatctttactgcggctatactcctgcgacgctaattgagcagctttttggtaagattgatcaaaaatggagagaaacggggccgaatggcgatgctactgtcatattcagatatgcaacaagtacaaataatttagttttctacaaaccgacgcagcttggacctacaggtgtaaaattacagtggagtcagttagataccgcttctggtggtggttttctttattgcaacagaagtgatagcacaagtggtagcgcaatgcgtattgaaaatgcaatggttgactcaggtaaaatgtatggctcccataaattatttaatacatcagttcctggtttgtattacacattattaatttcaaacatgtggtcagcttacggtaccgtaactaacgttagttcacctgggatatacattggtgactctgcagaacaatatttttcgtggtataatccaagcgaagacgtgttatattggagttgcaataatgcgaatagcacccgtaaatactgggctgtaggtggtatttatcagacccttacaattgaattctatacagatacaaactttgatccatatcaggagagcatt

SEQ ID NO: 3 - DNA, 1574 bp, Escherichia coli
atccacgttttgtattagcaaacgtcaaactctcatcgctgacagaactcaccgcaaaagaccttctcggttaatgaccaggggcagtgatcgtctcatggccttgccatggtgttctctatgttgctggcggcgattatctggaacctgggtacctggtactttggtttacctgcatccagctctcatacgctgattggcgcgatcgcttctaaagcagcccgcatgcgttccatcgtcgttcctgcgccagaagcgcaaaatgatcggccagcccaccaggaagcccagcgagtaaattaagccgtcatagccggaggtaaacaccagcgcggagatctcatttttaaccgcgtcaatcagcattgaagagtcctggcttaagtcatagcccggcggattaaccacctgcatttccagttcaataccgagggtaaaaggttcagaaacatgaaaatcgggtaatggcataggtttctcttaagttggcgttttctattcagtatagaagtcggagcggctgggcgagatgcggaagttctggaatgtttctttttttggtgatggtgactgaagcaatttggctacttttgcaatgtgacaagttatggcacggctggctggtggcgaagaattttgacgattgaggcatgcagaaaaaaaacgggttcagctttcagttgatcctcccagaactttgctctggggggatacggtccccgctgttccccgtcgcttaatctgcattatgccgcgtaactatggcgcggcgtttaagtttccttgccgatagcggcggctggcagcgttggttctttgccggtattgcgattggtattagcgtgatcaaattccgctggcggttatctctggcccaacgtttgcgaaagaactggcggcaggtttacctacagctatttcgctggcctcgaccgatcaggaatgcccagtgttgtattcagacgtccacgtgacttattaaagatctttactgcggctatactcctgcgacgctaattgagcagctttttggtaagattgatcaaaaatggagagaaacggggccgaatggcgatgctactgtcatattcagatatgcaacaagtacaaataatttagttttctacaaaccgacgcagcttggacctacaggtgtaaaattacagtggagtcagttagataccgcttctggtggtggttttctttattgcaacagaagtgatagcacaagtggtagcgcaatgcgtattgaaaatgcaatggttgactcaggtaaaatgtatggctcccataaattatttaatacatcagttcctggtttgtattacacattattaatttcaaacatgtggtcagcttacggtaccgtaactaacgttagttcacctgggatatacattggtgactctgcagaacaatatttttcgtggtataatccaagcgaagacgtgttatattggagttgcaataatgcgaatagcacccgtaaatactgggctgtaggtggtatttatcagacccttacaattgaattctatacagatacaaactttgatccatatcaggagagcatt

SEQ ID NO: 4 - DNA, 1000 bp, Escherichia coli
accgatttactgtttgttggccttgtgcaactcaatgatgtggagtcattgaaaatgattcagcgcagcagtgagctaacacaacgcctgaaatgatgcataaagcagcgactggattgagattttcctgaattagtgagctgatccgcagcaatattttgtttatcctgtattttcagagggaatggagtgtaacgctctgtattaacaaggagagcattaaaatgggtaaactcacgggcaagacagcactgattacgggcgcattgcagggaattggcgaaggaattgccagaacttttgcacgtcatggcgcgaacctaatcttgctggatatctcccctgagatcgaaaagctggcggacgaactgtgtggtcgtggtcatcgctgtacggcggttgtcgccgatgtgcgtgacccggcgtcggtagccgcagctatcaaacgcgcgaaggaaaaagaagggcgcattgatatcctggtgaataacgcaggcgtttgtcgtctgggcagtttcctcgatatgagcgatgacgatcgcgatttccatattgacatcaatattaaaggcgtatggaacgtcacgaaggcggtgctgccggagatgattgcccgcaaagatggtcgcattgtgatgatgtcttcagtcactggtgatatggtggccgatcctggcgaacaggcgtacgccttaacgaaagcggcgattgttggcctgacaaaatcgctggcggtggagtacgcgcagtctggtattcgcgttaacgccatttgcccgggatacgtgcgcacaccaatggcggaaagcattgcccgccagtcgaacccggaagatccagagtcggtgctgactgaaatggcgaaagcaatcccgatgcgtcgcctcgccgatccgctggaagtcggcgaactggcggccttcctcgcatcggatgaatccagctatttaaccggtacacagaatgtgattgatggcggcagcacactgccggagacggt

SEQ ID NO: 5 - protein, 263 aa, Escherichia coli
MGKLTGKTALITGALQGIGEGIARTFARHGANLILLDISPEIEKLADELCGRGHRCTAVVADVRDPASVAAAIKRAKEKEGRIDILVNNAGVCRLGSFLDMSDDDRDFHIDINIKGVWNVTKAVLPEMIARKDGRIVMMSSVTGDMVADPGEQAYALTKAAIVGLTKSLAVEYAQSGIRVNAICPGYVRTPMAESIARQSNPEDPESVLTEMAKAIPMRRLADPLEVGELAAFLASDESSYLTGTQNVIDGGSTLPETVSVGI

SEQ ID NO: 6 - DNA, 1149 bp, Escherichia coli
ttaccaggcggtatggtaaagctctacaatatcctcaagcgttgcttcacgcgggttgccaccggtacaaacatcatccagtgccgcctgagccagtgccggaatgtcttccttgcgtacaccaacatcacgcaaatgtggcggaataccgacatcacggttgagagcaaacaccgcttcaacagcggcattacgcgcctcttccaggctcataccttccactttcacgcccataacgcgcgcgatatcgcggtacttctcaccggtaaagtcagcgttatagcgcatgacatgcggtaacaggatggcgttcgcaacaccgtgtggagtgttgtaaaacgcgcccagtggatgcgccataccatgcaccaaccctaacccaacattcgagaagcccatacccgcaacatactgcccgagcgccatttcttctccggcatccttatcaccagcaaccgatcctcgcagcgccccagcaatgatttcaatcgctttaatgtgcagtgcatcggttagcgcccacgcgccacgggtaatatacccctcaatagcatgagtgagcgcatcgacacccgtcgcagctttcagcgctggaggcataccatccatcatgtcagcgtcaataaacgccacctgcgggatatcatgcggatcaacgcaaacaaacttgcgccgtttttcttcgtcagtgatcacgtagttaatggtcacttctgccgcagtgcctgctgtggtggggattgccagaatcggtacactgggtttattggtcggggaaagtccttccaggctacgcacatcggcaaactccgggttgttgctgataatgccaatcgctttacaagtatcctgtggagaaccaccaccaatagcgatcaggtaatccgcgccgctattctggaatacaccgagcccttctttgacgacagtaattgttgggttgggcactacgccgtcgtaaatcgcccatgccagccctgcagcatccatcttatcggtcactttcgccaccacgccgcattgcaccagcgttttatcggtgacgatcagcgccttctgataaccacggcgtttcacctcatcggttaaagccccaacagcaccccgaccaaaccatgccgtttcgttcagaatcattctgttagccat

SEQ ID NO: 7 - protein, 382 aa, Escherichia coli
MANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 8 - DNA, 1164 bp, Artificial sequence
  yqhD nucleic acid sequence
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 9 - protein, 387 aa, Artificial sequence
  yqhD amino acid sequence
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 10 - DNA, 1533 bp, Artificial sequence
  pntA nucleic acid
atgcgaattggcataccaagagaacggttaaccaatgaaacccgtgttgcagcaacgccaaaaacagtggaacagctgctgaaactgggttttaccgtcgcggtagagagcggcgcgggtcaactggcaagttttgacgataaagcgtttgtgcaagcgggcgctgaaattgtagaagggaatagcgtctggcagtcagagatcattctgaaggtcaatgcgccgttagatgatgaaattgcgttactgaatcctgggacaacgctggtgagttttatctggcctgcgcagaatccggaattaatgcaaaaacttgcggaacgtaacgtgaccgtgatggcgatggactctgtgccgcgtatctcacgcgcacaatcgctggacgcactaagctcgatggcgaacatcgccggttatcgcgccattgttgaagcggcacatgaatttgggcgcttctttaccgggcaaattactgcggccgggaaagtgccaccggcaaaagtgatggtgattggtgcgggtgttgcaggtctggccgccattggcgcagcaaacagtctcggcgcgattgtgcgtgcattcgacacccgcccggaagtgaaagaacaagttcaaagtatgggcgcggaattcctcgagctggattttaaagaggaagctggcagcggcgatggctatgccaaagtgatgtcggacgcgttcatcaaagcggaaatggaactctttgccgcccaggcaaaagaggtcgatatcattgtcaccaccgcgcttattccaggcaaaccagcgccgaagctaattacccgtgaaatggttgactccatgaaggcgggcagtgtgattgtcgacctggcagcccaaaacggcggcaactgtgaatacaccgtgccgggtgaaatcttcactacggaaaatggtgtcaaagtgattggttataccgatcttccgggccgtctgccgacgcaatcctcacagctttacggcacaaacctcgttaatctgctgaaactgttgtgcaaagagaaagacggcaatatcactgttgattttgatgatgtggtgattcgcggcgtgaccgtgatccgtgcgggcgaaattacctggccggcaccgccgattcaggtatcagctcagccgcaggcggcacaaaaagcggcaccggaagtgaaaactgaggaaaaatgtacctgctcaccgtggcgtaaatacgcgttgatggcgctggcaatcattctttttggctggatggcaagcgttgcgccgaaagaattccttgggcacttcaccgttttcgcgctggcctgcgttgtcggttattacgtggtgtggaatgtatcgcacgcgctgcatacaccgttgatgtcggtcaccaacgcgatttcagggattattgttgtcggagcactgttgcagattggccagggcggctgggttagcttccttagttttatcgcggtgcttatagccagcattaatattttcggtggcttcaccgtgactcagcgcatgctgaaaatgttccgcaaaaattaa

SEQ ID NO: 11 - protein, 510 aa, Artificial sequence
  pntA amino acid sequence
MRIGIPRERLTNETRVAATPKTVEQLLKLGFTVAVESGAGQLASFDDKAFVQAGAEIVEGNSVWQSEIILKVNAPLDDEIALLNPGTTLVSFIWPAQNPELMQKLAERNVTVMAMDSVPRISRAQSLDALSSMANIAGYRAIVEAAHEFGRFFTGQITAAGKVPPAKVMVIGAGVAGLAAIGAANSLGAIVRAFDTRPEVKEQVQSMGAEFLELDFKEEAGSGDGYAKVMSDAFIKAEMELFAAQAKEVDIIVTTALIPGKPAPKLITREMVDSMKAGSVIVDLAAQNGGNCEYTVPGEIFTTENGVKVIGYTDLPGRLPTQSSQLYGTNLVNLLKLLCKEKDGNITVDFDDVVIRGVTVIRAGEITWPAPPIQVSAQPQAAQKAAPEVKTEEKCTCSPWRKYALMALAIILFGWMASVAPKEFLGHFTVFALACVVGYYVVWNVSHALHTPLMSVTNAISGIIVVGALLQIGQGGWVSFLSFIAVLIASINIFGGFTVTQRMLKMFRKN

SEQ ID NO: 12 - DNA, 400 bp, Artificial sequence
  nucleic acid containing adhE promoter sequence
gacagcatttttcacctcctaactacttaaaattgctatcattcgttattgttatctagttgtgcaaaacatgctaatgtagccaccaaatcatactacaatttattaactgttagctataatggcgaaaagcgatgctgaaaggtgtcagctttgcaaaaatttgatttggatcacgtaatcagtacccagaagtgagtaatcttgcttacgccacctggaagtgacgcattagagataataactctaatgtttaaactcttttagtaaatcacagtgagtgtgagcgcgagtaagcttttgattttcataggttaagcaaatcatcaccgcactgactatactctcgtattcgagcagatgatttactaaaaaagtttaacattatcaggagagcatt

SEQ ID NO: 13 - DNA, 3732 bp, Artificial sequence
  nucleic acid sequence for adhE::pntAB
gacagcatttttcacctcctaactacttaaaattgctatcattcgttattgttatctagttgtgcaaaacatgctaatgtagccaccaaatcatactacaatttattaactgttagctataatggcgaaaagcgatgctgaaaggtgtcagctttgcaaaaatttgatttggatcacgtaatcagtacccagaagtgagtaatcttgcttacgccacctggaagtgacgcattagagataataactctaatgtttaaactcttttagtaaatcacagtgagtgtgagcgcgagtaagcttttgattttcataggttaagcaaatcatcaccgcactgactatactctcgtattcgagcagatgatttactaaaaaagtttaacattatcaggagagcattatgcgaattggcataccaagagaacggttaaccaatgaaacccgtgttgcagcaacgccaaaaacagtggaacagctgctgaaactgggttttaccgtcgcggtagagagcggcgcgggtcaactggcaagttttgacgataaagcgtttgtgcaagcgggcgctgaaattgtagaagggaatagcgtctggcagtcagagatcattctgaaggtcaatgcgccgttagatgatgaaattgcgttactgaatcctgggacaacgctggtgagttttatctggcctgcgcagaatccggaattaatgcaaaaacttgcggaacgtaacgtgaccgtgatggcgatggactctgtgccgcgtatctcacgcgcacaatcgctggacgcactaagctcgatggcgaacatcgccggttatcgcgccattgttgaagcggcacatgaatttgggcgcttctttaccgggcaaattactgcggccgggaaagtgccaccggcaaaagtgatggtgattggtgcgggtgttgcaggtctggccgccattggcgcagcaaacagtctcggcgcgattgtgcgtgcattcgacacccgcccggaagtgaaagaacaagttcaaagtatgggcgcggaattcctcgagctggattttaaagaggaagctggcagcggcgatggctatgccaaagtgatgtcggacgcgttcatcaaagcggaaatggaactctttgccgcccaggcaaaagaggtcgatatcattgtcaccaccgcgcttattccaggcaaaccagcgccgaagctaattacccgtgaaatggttgactccatgaaggcgggcagtgtgattgtcgacctggcagcccaaaacggcggcaactgtgaatacaccgtgccgggtgaaatcttcactacggaaaatggtgtcaaagtgattggttataccgatcttccgggccgtctgccgacgcaatcctcacagctttacggcacaaacctcgttaatctgctgaaactgttgtgcaaagagaaagacggcaatatcactgttgattttgatgatgtggtgattcgcggcgtgaccgtgatccgtgcgggcgaaattacctggccggcaccgccgattcaggtatcagctcagccgcaggcggcacaaaaagcggcaccggaagtgaaaactgaggaaaaatgtacctgctcaccgtggcgtaaatacgcgttgatggcgctggcaatcattctttttggctggatggcaagcgttgcgccgaaagaattccttgggcacttcaccgttttcgcgctggcctgcgttgtcggttattacgtggtgtggaatgtatcgcacgcgctgcatacaccgttgatgtcggtcaccaacgcgatttcagggattattgttgtcggagcactgttgcagattggccagggcggctgggttagcttccttagttttatcgcggtgcttatagccagcattaatattttcggtggcttcaccgtgactcagcgcatgctgaaaatgttccgcaaaaattaaggggtaacatatgtctggaggattagttacagctgcatacattgttgccgcgatcctgtttatcttcagtctggccggtctttcgaaacatgaaacgtctcgccagggtaacaacttcggtatcgccgggatggcgattgcgttaatcgcaaccatttttggaccggatacgggtaatgttggctggatcttgctggcgatggtcattggtggggcaattggtatccgtctggcgaagaaagttgaaatgaccgaaatgccagaactggtggcgatcctgcatagcttcgtgggtctggcggcagtgctggttggctttaacagctatctgcatcatgacgcgggaatggcaccgattctggtcaatattcacctgacggaagtgttcctcggtatcttcatcggggcggtaacgttcacgggttcggtggtggcgttcggcaaactgtgtggcaagatttcgtctaaaccattgatgctgccaaaccgtcacaaaatgaacctggcggctctggtcgtttccttcctgctgctgattgtatttgttcgcacggacagcgtcggcctgcaagtgctggcattgctgataatgaccgcaattgcgctggtattcggctggcatttagtcgcctccatcggtggtgcagatatgccagtggtggtgtcgatgctgaactcgtactccggctgggcggctgcggctgcgggctttatgctcagcaacgacctgctgattgtgaccggtgcgctggtcggttcttcgggggctatcctttcttacattatgtgtaaggcgatgaaccgttcctttatcagcgttattgcgggtggtttcggcaccgacggctcttctactggcgatgatcaggaagtgggtgagcaccgcgaaatcaccgcagaagagacagcggaactgctgaaaaactcccattcagtgatcattactccggggtacggcatggcagtcgcgcaggcgcaatatcctgtcgctgaaattactgagaaattgcgcgctcgtggtattaatgtgcgtttcggtatccacccggtcgcggggcgtttgcctggacatatgaacgtattgctggctgaagcaaaagtaccgtatgacatcgtgctggaaatggacgagatcaatgatgactttgctgataccgataccgtactggtgattggtgctaacgatacggttaacccggcggcgcaggatgatccgaagagtccgattgctggtatgcctgtgctggaagtgtggaaagcgcagaacgtgattgtctttaaacgttcgatgaacactggctatgctggtgtgcaaaacccgctgttcttcaaggaaaacacccacatgctgtttggtgacgccaaagccagcgtggatgcaatcctgaaagctctgtaatcagtagcgctgtctggcaacataaacggccccttctgggcaatgccgatcagttaaggattagttgaccgatccttaaactgaggcactataacggcttccacaacagggagccgttttcttatgccacttctcaatgatctgctcgatttcagtgaccatccgcttatgcctccgccctctgcacaactatttgcagaacaccttcccaccgagtggatacaacactgcctgacgctttctgctcatgcgaccgttcgccgccgtcgtttaccgggggacatggttatctggatggtggtgcaatgagccaattaccgatgttgttcgccgtctgaacctgagcgcggatggcgaagcggggatgaacctgctggcccgcagcgctgtcacccaggcg

SEQ ID NO: 14 - DNA, 3007 bp, Artificial sequence
  nucleic acid sequence for PyadC′fucO-ucpA
tgcgctacgctctatggctccctgacgtttttttagccaggaccacgtgacttattaaagatctttactgcggctatactcctgcgacgctaattgagcagctttttggtaagattgatcaaaaatggagagaaacggggccgaatggcgatgctactgtcatattcagatatgcaacaagtacaaataatttagttttctacaaaccgacgcagcttggacctacaggtgtaaaattacagtggagtcagttagataccgcttctggtggtggttttctttattgcaacagaagtgatagcacaagtggtagcgcaatgcgtattgaaaatgcaatggttgactcaggtaaaatgtatggctcccataaattatttaatacatcagttcctggtttgtattacacattattaatttcaaacatgtggtcagcttacggtaccgtaactaacgttagttcacctgggatatacattggtgactctgcagaacaatatttttcgtggtataatccaagcgaagacgtgttatattggagttgcaataatgcgaatagcacccgtaaatactgggctgtaggtggtatttatcagacccttacaattgaattctatacagatacaaactttgatccatatcaggagagcattatgatggctaacagaatgattctgaacgaaacggcatggtttggtcggggtgctgttggggctttaaccgatgaggtgaaacgccgtggttatcagaaggcgctgatcgtcaccgataaaacgctggtgcaatgcggcgtggtggcgaaagtgaccgataagatggatgctgcagggctggcatgggcgatttacgacggcgtagtgcccaacccaacaattactgtcgtcaaagaagggctcggtgtattccagaatagcggcgcggattacctgatcgctattggtggtggttctccacaggatacttgtaaagcgattggcattatcagcaacaacccggagtttgccgatgtgcgtagcctggaaggactttccccgaccaataaacccagtgtaccgattctggcaatccccaccacagcaggcactgcggcagaagtgaccattaactacgtgatcactgacgaagaaaaacggcgcaagtttgtttgcgttgatccgcatgatatcccgcaggtggcgtttattgacgctgacatgatggatggtatgcctccagcgctgaaagctgcgacgggtgtcgatgcgctcactcatgctattgaggggtatattacccgtggcgcgtgggcgctaaccgatgcactgcacattaaagcgattgaaatcattgctggggcgctgcgaggatcggttgctggtgataaggatgccggagaagaaatggcgctcgggcagtatgttgcgggtatgggcttctcgaatgttgggttagggttggtgcatggtatggcgcatccactgggcgcgttttacaacactccacacggtgttgcgaacgccatcctgttaccgcatgtcatgcgctataacgctgactttaccggtgagaagtaccgcgatatcgcgcgcgttatgggcgtgaaagtggaaggtatgagcctggaagaggcgcgtaatgccgctgttgaagcggtgtttgctctcaaccgtgatgtcggtattccgccacatttgcgtgatgttggtgtacgcaaggaagacattccggcactggctcaggcggcactggatgatgtttgtaccggtggcaacccgcgtgaagcaacgcttgaggatattgtagagctttaccataccgcctggtaaaattgaagaaggaataaggtatgggtaaactcacgggcaagacagcactgattacgggcgcattgcagggaattggcgaaggaattgccagaacttttgcgcgtcatggcgcaaacctaatcttgctggatatctcccctgagatcgaaaagctggcggacgaactgtgtggtcgtggtcatcgctgtacggcggttgtcgccgatgtgcgtgacccggcgtcggtagccgcagctatcaaacgcgcgaaggaaaaagaagggcgcattgatatcctggtgaataacgcaggcgtttgtcgtctgggcagtttcctcgatatgagcgatgaagatcgcgatttccatattgacatcaatattaaaggcgtatggaacgtcacgaaggcggtgctgccagagatgattgcgcgcaaagatggtcgcattgtgatgatgtcttcagtcactggtgatatggtggccgatcctggcgaaacggcgtacgccttaacgaaagcggcgattgttggcctgacaaaatcgctggcggtggagtacgcgcagtctggtattcgcgttaacgccatttgcccgggatacgtgcgcacaccaatggcggaaagcattgcccgccagtcgaacccggaagatccagagtcggtgctgactgaaatggcgaaagcaatcccgatgcgtcgcctcgccgatccgctggaagtcggcgaactggcggccttcctcgcatcggatgaatccagctatttaaccggtacacagaatgtgattgatggcggcagcacactgccggagacggttagcgtcggtatctgatttcacaccgccagctcagctggcggtgctgttttgtaacccgccaaatcggcggtaacgaaagaggataaaccgtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgtttaat

SEQ ID NO: 15 - DNA, 20 bp, Artificial sequence
  intergenic sequence
aattgaagaaggaataaggt

SEQ ID NO: 16 - DNA, 1777 bp, Escherichia coli
  misc_feature (1)..(178): ackA′ upstream sequence
tacttgagtcgtcaaattcatatacattatgccattggctgaaaattacgcaaaatggcatagactcaagatatttcttccatcatgcaaaaaaaatttgcagtgcatgatgttaatcataaatgtcggtgtcatcatgcgctacgctctatggctccctgacgtttttttagccaggatccacgttttgtattagcaaacgtcaaactctcatcgctgcaaatcaccgcaaaagaccttctcggttaatgaccaggggcagtgatcgtctcatggccttgccatggtgttctctatgttgctggcggcgattatctggaacctgggtacctggtactttggtttacctgcatccagctctcatacgctgattggcgcgatcgcttctaaagcagcccgcatgcgttccatcgtcgttcctgcgccagaagcgcaaaatgatcggccagcccaccaggaagcccagcgagtaaattaagccgtcatagccggaggtaaacaccagcgcggagatctcatttttaaccgcgtcaatcagcattgaagagtcctggcttaagtcatagcccggcggattaaccacctgcatttccagttcaataccgagggtaaaaggttcagaaacatgaaaatcgggtaatggcataggtttctcttaagttggcgttttctattcagtatagaagtcggagcggctgggcgagatgcggaagttctggaatgtttctttttttggtgatggtgactgaagcaatttggctacttttgcaatgtgacaagttatggcacggctggctggtggcgaagaattttgacgattgaggcatgcagaaaaaaaacgggttcagctttcagttgatcctcccagaactttgctctggggggatacggtccccgctgttccccgtcgcttaatctgcattatgccgcgtaactatggcgcggcgtttaagtttccttgccgatagcggcggctggcagcgttggttctttgccggtattgcgattggtattagcgtgatcaaattccgctggcggttatctctggcccaacgtttgcgaaagaactggcggcaggtttacctacagctatttcgctggcctcgaccgatcaggaatgcccagtgttgtattcagacgtccacgtgacttattaaagatctttactgcggctatactcctgcgacgctaattgagcagctttttggtaagattgatcaaaaatggagagaaacggggccgaatggcgatgctactgtcatattcagatatgcaacaagtacaaataatttagttttctacaaaccgacgcagcttggacctacaggtgtaaaattacagtggagtcagttagataccgcttctggtggtggttttctttattgcaacagaagtgatagcacaagtggtagcgcaatgcgtattgaaaatgcaatggttgactcaggtaaaatgtatggctcccataaattatttaatacatcagttcctggtttgtattacacattattaatttcaaacatgtggtcagcttacggtaccgtaactaacgttagttcacctgggatatacattggtgactctgcagaacaatatttttcgtggtataatccaagcgaagacgtgttatattggagttgcaataatgcgaatagcacccgtaaatactgggctgtaggtggtatttatcagacccttacaattgaattctatacagatacaaactttgatccatatcaggagagcattatgatggctaacagaatgattctgaacg

SEQ ID NO: 17 - DNA, 808 bp, Escherichia coli
  misc_feature (1)..(171): ackA′ upstream sequence
gtcgtcaaattcatatacattatgccattggctgaaaattacgcaaaatggcatagactcaagatatttcttccatcatgcaaaaaaaatttgcagtgcatgatgttaatcataaatgtcggtgtcatcatgcgctacgctctatggctccctgacgtttttttagccaggaccacgtgacttattaaagatctttactgcggctatactcctgcgacgctaattgagcagctttttggtaagattgatcaaaaatggagagaaacggggccgaatggcgatgctactgtcatattcagatatgcaacaagtacaaataatttagttttctacaaaccgacgcagcttggacctacaggtgtaaaattacagtggagtcagttagataccgcttctggtggtggttttctttattgcaacagaagtgatagcacaagtggtagcgcaatgcgtattgaaaatgcaatggttgactcaggtaaaatgtatggctcccataaattatttaatacatcagttcctggtttgtattacacattattaatttcaaacatgtggtcagcttacggtaccgtaactaacgttagttcacctgggatatacattggtgactctgcagaacaatatttttcgtggtataatccaagcgaagacgtgttatattggagttgcaataatgcgaatagcacccgtaaatactgggctgtaggtggtatttatcagacccttacaattgaattctatacagatacaaactttgatccatatcaggagagcattatgatggctaacagaatgattctgaacg

SEQ ID NO: 18 - DNA, 20 bp, Artificial Sequence
  yqhD up
tatgatgccaggctcgtaca

SEQ ID NO: 19 - DNA, 20 bp, Artificial Sequence
  yqhD Down
gatcatgcctttccatgctt

SEQ ID NO: 20 - DNA, 24 bp, Artificial Sequence
  yqhD 1
gctttttacgcctcaaactttcgt

SEQ ID NO: 21 - DNA, 19 bp, Artificial Sequence
  yqhD 2
tacttgctccctttgctgg

SEQ ID NO: 22 - DNA, 20 bp, Artificial Sequence
  adhE up
caatacgccttttgacagca

SEQ ID NO: 23 - DNA, 20 bp, Artificial Sequence
  adhE down
gccatcaatggcaaaaagtt

SEQ ID NO: 24 - DNA, 56 bp, Artificial Sequence
  adhE-pntAB ORF up
tactaaaaaagtttaacattatcaggagagcattatgcgaattggcataccaagag

SEQ ID NO: 25 - DNA, 41 bp, Artificial Sequence
  adhE-pntAB ORF down
tgccagacagcgctactgattacagagctttcaggattgca

SEQ ID NO: 26 - DNA, 41 bp, Artificial Sequence
  adhE-pntAB 1
tgcaatcctgaaagctctgtaatcagtagcgctgtctggca

SEQ ID NO: 27 - DNA, 56 bp, Artificial Sequence
  adhE-pntAB 2
ctcttggtatgccaattcgcataatgctctcctgataatgttaaacttttttagta

SEQ ID NO: 28 - DNA, 64 bp, Artificial Sequence
  pTrcFucO-UcpA left
cttgcccgtgagtttacccataccttattccttcttcaattttaccaggcggtatggtaaagct

SEQ ID NO: 29 - DNA, 45 bp, Artificial Sequence
  pTrcFucO-UcpA right
cggttagcgtcggtatctgaatgcgctgatgtgataatgccggat

SEQ ID NO: 30 - DNA, 41 bp, Artificial Sequence
  pTrcFucO-UcpA ORFup
aattgaagaaggaataaggtatgggtaaactcacgggcaag

SEQ ID NO: 31 - DNA, 45 bp, Artificial Sequence
  pTrcFucO-UcpA ORF down
atccggcattatcacatcagcgcattcagataccgacgctaaccg

SEQ ID NO: 32 - DNA, 20 bp, Artificial Sequence
  ackA 10
gactcttccggcatagtctg

SEQ ID NO: 33 - DNA, 20 bp, Artificial Sequence
  ackA 20
gcatgagcgttgacgcaatc

SEQ ID NO: 34 - DNA, 20 bp, Artificial Sequence
  ackA up
ctggttctgaactgcggtag

SEQ ID NO: 35 - DNA, 20 bp, Artificial Sequence
  ackA down
cgcgataaccagttcttcgt

SEQ ID NO: 36 - DNA, 20 bp, Artificial Sequence
  ackAup 200
ttagcagcctgaaggcctaa

SEQ ID NO: 37 - DNA, 20 bp, Artificial Sequence
  ackAdown 200
acgacttcagcgtctttggt

SEQ ID NO: 38 - DNA, 20 bp, Artificial Sequence
  pACYC-up
cacctcgctaacggattcac

SEQ ID NO: 39 - DNA, 20 bp, Artificial Sequence
  pACYC-down
ggatgacgatgagcgcattg

SEQ ID NO: 40 - DNA, 20 bp, Artificial Sequence
  ackA 1
tttcacaccgccagctcagc

SEQ ID NO: 41 - DNA, 37 bp, Artificial Sequence
  ackA 2
ggaagtacctataattgatacgtggctaaaaaaacgt

SEQ ID NO: 42 - DNA, 45 bp, Artificial Sequence
  ackApAC up
gtatcaattataggtacttccatgatggctaacagaatgattctg

SEQ ID NO: 43 - DNA, 45 bp, Artificial Sequence
  ackApAC down
gctgagctggcggtgtgaaatcagataccgacgctaaccgtctcc

SEQ ID NO: 44 - DNA, 32 bp, Artificial Sequence
  pACYC PacI
gcatttaattaacctgtggaacacctacatct

SEQ ID NO: 45 - DNA, 20 bp, Artificial Sequence
  pACYC HindIII
aaccaagcctatgcctacag

SEQ ID NO: 46 - DNA, 45 bp, Artificial Sequence
  HindIII ackA fucO
gcataagcttttagcagcctgaaggcctaagtagtacatattcat

SEQ ID NO: 47 - DNA, 41 bp, Artificial Sequence
  ackA fucO PacI
gcatttaattaaacgacttcagcgtctttggtgttagcgtg

SEQ ID NO: 48 - DNA, 48 bp, Artificial Sequence
  fucO RBS
tatcaggagagcattatgatggctaacagaatgattctgaacgaaacg

SEQ ID NO: 49 - DNA, 51 bp, Artificial Sequence
  fucO BamHI
ggatcctggctaaaaaaacgtcagggagccatagagcgtagcgcatgatga

SEQ ID NO: 50 - DNA, 61 bp, Artificial Sequence
  adhE-fucO ORF up
tactaaaaaagtttaacattatcaggagagcattatgatggctaacagaatgattctgaac

SEQ ID NO: 51 - DNA, 40 bp, Artificial Sequence
  adhE-fucO ORF down
tgccagacagcgctactgattaccaggcggtatggtaaag

SEQ ID NO: 52 - DNA, 40 bp, Artificial Sequence
  adhE-fucO 1
ctttaccataccgcctggtaatcagtagcgctgtctggca

SEQ ID NO: 53 - DNA, 61 bp, Artificial Sequence
  adhE-fucO 2
gttcagaatcattctgttagccatcataatgctctcctgataatgttaaacttttttagta

SEQ ID NO: 54 - DNA, 20 bp, Artificial Sequence
  fucO ORF left
accagcgttttatcggtgac

SEQ ID NO: 55 - DNA, 20 bp, Artificial Sequence
  ackA up 200
ttagcagcctgaaggcctaa

SEQ ID NO: 56 - DNA, 1389 bp, Artificial sequence
  pntB nucleic acid sequence
atgtctggaggattagttacagctgcatacattgttgccgcgatcctgtttatcttcagtctggccggtctttcgaaacatgaaacgtctcgccagggtaacaacttcggtatcgccgggatggcgattgcgttaatcgcaaccatttttggaccggatacgggtaatgttggctggatcttgctggcgatggtcattggtggggcaattggtatccgtctggcgaagaaagttgaaatgaccgaaatgccagaactggtggcgatcctgcatagcttcgtgggtctggcggcagtgctggttggctttaacagctatctgcatcatgacgcgggaatggcaccgattctggtcaatattcacctgacggaagtgttcctcggtatcttcatcggggcggtaacgttcacgggttcggtggtggcgttcggcaaactgtgtggcaagatttcgtctaaaccattgatgctgccaaaccgtcacaaaatgaacctggcggctctggtcgtttccttcctgctgctgattgtatttgttcgcacggacagcgtcggcctgcaagtgctggcattgctgataatgaccgcaattgcgctggtattcggctggcatttagtcgcctccatcggtggtgcagatatgccagtggtggtgtcgatgctgaactcgtactccggctgggcggctgcggctgcgggctttatgctcagcaacgacctgctgattgtgaccggtgcgctggtcggttcttcgggggctatcctttcttacattatgtgtaaggcgatgaaccgttcctttatcagcgttattgcgggtggtttcggcaccgacggctcttctactggcgatgatcaggaagtgggtgagcaccgcgaaatcaccgcagaagagacagcggaactgctgaaaaactcccattcagtgatcattactccggggtacggcatggcagtcgcgcaggcgcaatatcctgtcgctgaaattactgagaaattgcgcgctcgtggtattaatgtgcgtttcggtatccacccggtcgcggggcgtttgcctggacatatgaacgtattgctggctgaagcaaaagtaccgtatgacatcgtgctggaaatggacgagatcaatgatgactttgctgataccgataccgtactggtgattggtgctaacgatacggttaacccggcggcgcaggatgatccgaagagtccgattgctggtatgcctgtgctggaagtgtggaaagcgcagaacgtgattgtctttaaacgttcgatgaacactggctatgctggtgtgcaaaacccgctgttcttcaaggaaaacacccacatgctgtttggtgacgccaaagccagcgtggatgcaatcctgaaagctctgtaa

SEQ ID NO: 57 - protein, 462 aa, Artificial sequence
  pntB amino acid sequence
MSGGLVTAAYIVAAILFIFSLAGLSKHETSRQGNNFGIAGMAIALIATIFGPDTGNVGWILLAMVIGGAIGIRLAKKVEMTEMPELVAILHSFVGLAAVLVGFNSYLHHDAGMAPILVNIHLTEVFLGIFIGAVTFTGSVVAFGKLCGKISSKPLMLPNRHKMNLAALVVSFLLLIVFVRTDSVGLQVLALLIMTAIALVFGWHLVASIGGADMPVVVSMLNSYSGWAAAAAGFMLSNDLLIVTGALVGSSGAILSYIMCKAMNRSFISVIAGGFGTDGSSTGDDQEVGEHREITAEETAELLKNSHSVIITPGYGMAVAQAQYPVAEITEKLRARGINVRFGIHPVAGRLPGHMNVLLAEAKVPYDIVLEMDEINDDFADTDTVLVIGANDTVNPAAQDDPKSPIAGMPVLEVWKAQNVIVFKRSMNTGYAGVQNPLFFKENTHMLFGDAKASVDAILKAL

(19) United States
(12) Patent Application Publication
(10) Pub. No.: US 2015/0299670 A1
(43) Pub. Date: Oct. 22, 2015

(54) COMBINING GENETIC TRAITS FOR FURFURAL TOLERANCE

(71) Applicant: UNIVERSITY OF FLORIDA RESEARCH FOUNDATION INCORPORATED, Gainesville, FL (US)

(72) Inventors: XUAN WANG, CHANDLER, AZ (US); LORRAINE P. YOMANO, GAINESVILLE, FL (US); JAMES Y. LEE, ORLANDO, FL (US); SEAN W. YORK, GAINESVILLE, FL (US); HUABAO ZHENG, GAINESVILLE, FL (US); MICHAEL TODD MULLINNIX, SARASOTA, FL (US); KEELNATHAM T. SHANMUGAM, GAINESVILLE, FL (US); LONNIE O'NEAL INGRAM, GAINESVILLE, FL (US)

(21) Appl. No.: 14/443,108

(22) PCT Filed: Nov. 14, 2013
(86) PCT No.: PCT/US13/69987

§ 371 Date: May 15, 2015

Related U.S. Application Data
(60) Provisional application No. 61/727,360, filed on Nov. 16, 2012.

(51) Int. Cl. C12N 9/04; C12N 15/70

(52) U.S. Cl. CPC C12N 9/0006; C12N 15/70; C12Y 101/01077

(57) ABSTRACT

Four genetic traits have been identified that increase furfural tolerance in microorganisms, such as ethanol-producing Escherichia coli LY180 (strain W derivative). Increased expression of fucO, ucpA or pntAB, and deletion of yqhD were associated with the increase in furfural tolerance. Microorganisms engineered for resistance to furfural were also more resistant to the mixture of inhibitors in hemicellulose hydrolysates, confirming the importance of furfural as an inhibitory component. The combinations of genetic traits disclosed in this application can be applied, generally, to other microorganisms, such as Gram negative and Gram positive bacterial cells, yeast and fungi to increase furfural tolerance in microorganisms used to make industrially useful products.

### CROSS-REFERENCE TO RELATED APPLICATION

This application claims the benefit of U.S. Provisional Application Ser. No. 61/727,360, filed Nov. 16, 2012, the disclosure of which is hereby incorporated by reference in its entirety, including all figures, tables and amino acid or nucleic acid sequences.

This invention was made with government support under 2011-10006-30358 awarded by the U.S. Department of Agriculture. The government has certain rights in the invention.

### BACKGROUND OF THE INVENTION

The carbohydrate component of lignocellulose represents a potential feedstock for renewable fuels and chemicals (1-3), an alternative to food crops and petroleum. However, the cost-effective use of lignocellulosic sugars in fermentation remains challenging (4, 5). Unlike starch, lignocellulose has been designed by nature to resist deconstruction (2, 6). Crystalline fibers of cellulose are encased in a covalently linked mesh of lignin and hemicellulose. Steam pretreatment with dilute mineral acids is an efficient approach to depolymerize hemicellulose (20-40% of biomass dry weight) into sugars (hemicellulose hydrolysate, primarily xylose) and to increase the access of cellulase enzymes (2, 3, 6). However, side reaction products (furfural, 5-hydroxymethylfurfural, formate, acetate, and soluble lignin products) are formed during pretreatment that hinder fermentation (7, 8). Furfural (dehydration product of pentose sugars) is widely regarded as one of the most important inhibitors (6-8). The concentration of furfural is correlated with the toxicity of dilute acid hydrolysates (9). Although overliming to pH 10 with Ca(OH)₂ can be used to reduce the level of furfural and toxicity, inclusion of this step increases process complexity and costs (9, 10).

Escherichia coli and yeasts have proven to be excellent biocatalysts for metabolic engineering (11, 12). However, both are inhibited by furans (7, 8, 13-15) and both contain NADPH-dependent oxidoreductases that convert furfural and hydroxymethylfurfural (dehydration product of hexose sugars) into less toxic alcohols (15-17). It is this depletion of NADPH by oxidoreductases such as YqhD (low Kₘ for NADPH) that has been proposed as the mechanism for growth inhibition in E. coli (FIG. 1) (18, 19). Growth resumed only after the complete reduction of furfural (19). A similar furan-induced delay in growth has been reported for fermenting yeasts (14, 15). Independent mutants of E. coli selected for resistance to furfural and hemicellulose hydrolylsate were found to contain mutations that silenced yqhD expression (17, 20). The NADPH-intensive pathway for sulfate assimilation was identified as an early site affected by furfural (18). Addition of cysteine (18), deletion of yqhD (19) or increased expression of pntAB (transhydrogenase for interconversion of NADH and NADPH) increased tolerance to furan aldehydes (18, 21) (FIG. 1). Furfural tolerance was also increased by overexpression of an NADH-dependent propanediol (and furfural) oxidoreductase (fucO) normally used for fucose metabolism (17), and by overexpression of a cryptic gene (ucpA) adjacent to a sulfur assimilation operon (22) (FIG. 1). However, none of these traits alone fully eliminated the problem of furfural toxicity. There remains a need to improve the resistance of microorganisms to furfural and hydroxymethylfurfural toxicity. As disclosed herein, we have identified combinations of genetic modifications that provide bacterial strains that exhibit an increase in furfural tolerance and an increase in tolerance to toxins in hemicellulose hydrolysates.

### BRIEF SUMMARY OF THE INVENTION

Four genetic traits have been identified that increase furfural tolerance in microorganisms, such as ethanol-producing Escherichia coli LY180 (strain W derivative). Increased expression of fucO, ucpA or pntAB, and deletion of yqhD were associated with the increase in furfural tolerance. As a proof of concept, plasmids and integrated strains were used to characterize epistatic interactions among traits and to identify the most effective combinations. Furfural resistance traits were subsequently integrated into the chromosome of LY180 to construct strain XW129 (LY180 ΔyqhD ackA::P_{yadC′}fucO-ucpA) for ethanol production. This same combination of traits was also constructed in succinate biocatalysts (E. coli strain C derivatives) and found to increase furfural tolerance. Strains engineered for resistance to furfural were also more resistant to the mixture of inhibitors in hemicellulose hydrolysates, confirming the importance of furfural as an inhibitory component. With resistant biocatalysts, product yields (ethanol and succinate) from hemicellulose syrups were equal to control fermentations in laboratory media without inhibitors. The combinations of genetic traits disclosed in this application can be applied, generally, to other microorganisms, such as Gram negative and Gram positive bacterial cells, yeast and fungi) to increase furfural tolerance in microorganisms used to make industrially useful products.

### BRIEF DESCRIPTION OF THE DRAWINGS

FIG. 1. Model showing relationships of furfural resistance traits, metabolism, and reducing cofactors. NADPH-linked reduction of furfural by YqhD is proposed to compete with biosynthesis, starving key steps in biosynthesis such as sulfate assimilation (18, 19). Deletion of yqhD or increased expression of pntAB (NADH/NADPH transhydrogenase) mitigated this problem by increasing the availability of NADPH. Overexpression of fucO increased the rate of furfural reduction and used NADH, an abundant cofactor during sugar fermentation (17). The cryptic gene ucpA is required for native furfural tolerance, and further increased furfural resistance when overexpressed (22).

FIGS. 2A-B. Epistatic interactions of furfural resistance traits during ethanol production. Fermentations were conducted in AM1 mineral salts medium (100 g/L xylose, 0.1 mM IPTG and 12.5 mg/L ampicillin) with 15 mM furfural. (A) Single furfural-resistant traits. LY180 containing empty vector pTrc99a (EV) was included as a control with and without furfural. LY180 ΔyqhD and LY180 adhE::pntAB also contained an empty vector to reduce differences related to plasmid burden. (B) Comparison of furfural tolerance for ethanol production (48 h). Test strains contain either empty vector or plasmids for expression of fucO, ucpA or fucO-ucpA. Ethanol titers of parent strain LY80 (hatched bars) were included with or without furfural for comparison. Modified strains contain a single trait (open/white bars), two traits (vertical bars), three traits (checker board bars) or four traits (black bar). Strain XW129 (LY180 ΔyqhD ackA::P_{yadC′}fucO-ucpA) was obtained after promoter engineering and chromosomal integration (horizontal bar). The 4 color boxes at the top of the figure represent a key to genetic traits. Stacked boxes correspond to traits in each respective strain. Data represent averages of at least 2 experiments with standard deviations.

FIGS. 3A-B. Comparison of in vitro furfural reductase activity and furfural resistance. NADH-linked furfural-dependent reductase activity (A) and furfural tolerance for growth (B) are shown for plasmid-free strains containing predicted optimal combinations of furfural resistance traits. Cell mass was measured from tube cultures (n=3) grown for 48 h in AM1 minimal media containing 50 g/L xylose with 12.5 mM furfural. Data represent averages of at least 2 experiments with standard deviations.

FIGS. 4A-C. Comparison of batch fermentations for the parent LY180 and the plasmid-free, furfural-resistant strain XW129. Furfural resistance traits in XW129 (LY180 ΔyqhD ackA::P_{yadC′}fucO-ucpA) improved fermentation with furfural in AM1 medium and also improved the fermentation of hemicellulose hydrolysate. For A (cell mass) and B (ethanol and furfural), fermentations were conducted in mineral salt medium AM1 (100 g/L xylose and 15 mM furfural). Control fermentations without furfural were also included. Fermentations (C) were also conducted using hemicellulose hydrolysate containing 36 g/L total sugar, supplemented with AM1 nutrients and 0.5 mM sodium metabisulfite. Data represent averages of at least 2 experiments with standard deviations.

FIGS. 5A-C. Engineering furfural-resistant derivatives of E. coli C for hemicellulose conversion to succinate. (A) Fermentation titer and yield (96 h) for parent KJ122 and mutant XW055 selected for improved xylose metabolism. Strains were grown in AM1 medium containing 100 g/L xylose as previously described (27) using KOH/K₂CO₃ to automatically maintain pH 7. Yield was calculated as g succinate produced per g xylose metabolized. (B) Comparison of furfural tolerance in tube cultures containing AM1 medium (50 g/L xylose, 100 mM MOPS, and 50 mM KHCO₃). Strain XW055 was compared to strains XW120 and XW136 containing chromosomally integrated traits for furfural resistance. Cell mass was measured after incubation for 48 h. (C) Fermentation of hemicellulose hydrolysate (AM1 nutrients, 0.5 mM sodium metabisulfite, 100 mM potassium bicarbonate, and 36 g/L total sugar). Strain XW136 (XW055 ΔyqhD ackA::P_{yadC′}fucO-ucpA adhE::fucO) completed the reduction of furfural in 24 h, coincident with the onset of rapid fermentation. Strain XW055 was unable to completely metabolize furfural or ferment sugars in hemicellulose hydrolysate. Data for furfural and succinate are shown by broken lines and solid lines, respectively. All data represent averages of at least 2 experiments with standard deviations.

FIGS. 6A-E. Isolation and characterization of the surrogate promoter for chromosomal expression of fucO-ucpA cassette.

(A) Promoter-probe plasmid pLOI4870 was used to isolate Sau3A1 fragments that serve as surrogate promoters for expression of fucO-ucpA. Two rounds of the growth-based screen were employed in AM1 medium containing furfural. (B) Isolation and identification of promoter fragment by sequencing pLOI5237 and pLOI5259. A putative promoter (boxed region) was predicted within this fragment using BPROM and Neural Network Promoter Prediction. (C) Growth of strains containing furfural-resistance plasmids expressing the fucO-ucpA cassette. Tube cultures (n=3) were grown for 48 h in AM1 medium containing 50 g/L xylose, 20 mg/L chloramphenicol and 12.5 or 15 mM furfural as previously described (2). (D) The NADH-linked furfural reductase activity in plasmid strains containing fucO-ucpA cassettes. (E) SDS-PAGE of cytoplasmic extracts from strains harboring fucO-ucpA cassettes. Arrows indicates the predicted size of FucO (MW 40.5 kDa; thick band) and UcpA (MW 27.8 kDa; not easily seen).

FIGS. 7A-B. Effects of furfural resistance traits in succinate-producing strains. Cell mass was measured from tube cultures (n=3) grown for 48 h in AM1 minimal media containing 50 g/L xylose with 10 mM (A) or 12.5 mM (B) furfural, 100 mM MOPS and 50 mM KHCO₃. Data represent averages of at least 3 experiments with standard deviations.

FIG. 8. Effect of plasmid-expressed fucO and ucpA on furfural tolerance of XW120 (XW055, ΔyqhD ackA::P_{yadC′}fucO-ucpA) during succinate production from xylose. Tube cultures (n=3) were grown for 48 h in AM1 medium containing 50 g/L xylose, 100 mM MOPS, 50 mM KHCO₃, 0.1 mM IPTG and 12.5 mg/L ampicillin with varying concentrations of furfural. Only plasmid pTrc fucO improved the furfural tolerance of strain XW120.

FIG. 9. Comparison of furfural resistance between strains XW055 and LY180. Cell mass was measured from tube cultures (n=3) grown for 48 h in AM1 minimal media containing 50 g/L xylose with varied concentrations of furfural (additional 100 mM MOPS and 50 mM KHCO₃ included for XW055). Data represent averages of at least 3 experiments with standard deviations. Cultures were inoculated to an initial density of 22 mg dry cell weight (dcw) per liter.

### BRIEF DESCRIPTION OF THE SEQUENCES

SEQ ID NO: 1: promoter sequence derived from E. coli.

SEQ ID NO: 2: E. coli DNA fragment containing promoter sequence (SEQ ID NO: 1).

SEQ ID NO: 3: E. coli DNA fragment containing promoter sequence (SEQ ID NO: 1).

SEQ ID NOs: 4-5: ucpA nucleic acid and amino acid sequences.

SEQ ID NOs: 6-7: fucO nucleic acid and amino acid sequences.

SEQ ID NOs: 8-9: yqhD nucleic acid and amino acid sequences.

SEQ ID NOs: 10-11: pntA nucleic acid and amino acid sequences.

SEQ ID NO: 12: adhE promoter sequence.

SEQ ID NO: 13: nucleic acid sequence for adhE::pntAB.

SEQ ID NO: 14: nucleic acid sequence for P_{yadC′}fucO-ucpA.

SEQ ID NOs: 56-57: pntB nucleic acid and amino acid sequences.

### DETAILED DISCLOSURE OF THE INVENTION

The invention provides organisms for production of renewable fuels and other chemicals. Particularly, the invention provides bacteria, fungi and yeast that can grow and produce renewable fuels and other chemicals in the presence of increased furfural. The invention provides for an isolated or recombinant cell/microorganism (bacterial, yeast or fungal cell) having increased expression of ucpA and fucO in combination with the deletion of the gene encoding yqhD or chromosomal integration of genes encoding pntA and pntB behind the adhE promoter (adhE::pntAB) and that exhibits improved ability to produce a desired product in the presence of furfural and 5-HMF as compared to a reference cell (e.g., a reference bacterial, yeast or fungal cell). In various embodiments, the bacterial, fungal or yeast cell contains a genetic construct comprising ucpA and fucO operably linked to a promoter comprising SEQ ID NO: 1. In various other embodiments, the bacterial, fungal or yeast cell may comprise, in addition to the aforementioned genetic modifications, a nucleic acid sequence encoding fucO that is integrated into the genome of the bacterial, fungal or yeast cell and operably linked to a native promoter within the genome of the bacterial, fungal or yeast cell (for example, the promoter for alcohol/acetaldehyde dehydrogenase (adhE)). In various embodiments, the bacterial, fungal or yeast cell having increased furfural and/or 5-HMF tolerance can produce ethanol; lactic acid; succinic acid; malic acid; acetic acid; 1,3-propanediol; 2,3-propanediol; pyruvate; dicarboxylic acids; adipic acid; butanol; and amino acids, including aliphatic and aromatic amino acids.

Various publications have disclosed bacterial, fungal or yeast cells in which ethanol; lactic acid; succinic acid; malic acid; acetic acid; 1,3-propanediol; 2,3-propanediol; 1,4-butanediol; 2,3-butanediol; butanol; pyruvate; dicarboxylic acids; adipic acid; and amino acids, including aliphatic and aromatic amino acids can be produced. Many of these microorganisms have been genetically manipulated (genetically engineered) in order to produce these desired products. Exemplary publications in this regard include U.S. Published Patent Applications US-2010/0184171A1 (directed to the production of malic acid and succinic acid), 2009/0148914A1 (directed to the production of acetic acid; 1,3-propanediol; 2,3-propanediol; pyruvate; dicarboxylic acids; adipic acid; and amino acids, including aliphatic and aromatic amino acids), 2007/0037265A1 (directed to the production of chirally pure D and L lactic acid) and PCT application PCT/US2010/029728 (published as WO2010/15067 and directed to the production of succinic acid). The teachings of each of these publications, with respect to the production of bacterial cells producing a desired product, is hereby incorporated by reference in its entirety.

In another aspect of the invention, bacterial, fungal or yeast cells disclosed herein demonstrate increased growth in the presence of furfural and/or 5-HMF as compared to a reference bacterial, fungal or yeast cell. In another embodiment, the bacterial, fungal or yeast cell has increased growth in the presence of furfural and/or 5-HMF at concentrations of about 5 mM, 10 mM, 15 mM, 20 mM, 25 mM, 30 mM, 35 mM, 40 mM or higher (or between about 5 mM and about 20 mM furfural and/or 5-HMF, about 15 mM to about 30 mM furfural and/or 5-HMF, preferably about 15 mM furfural and/or 5 HMF).

Bacterial cells can be selected Gram negative bacteria or Gram positive bacteria. In this aspect of the invention, the Gram-negative bacterial cell can be selected from the group consisting of Escherichia, Zymomonas, Acinetobacter, Gluconobacter, Geobacter, Shewanella, Salmonella, Enterobacter and Klebsiella. Gram-positive bacteria can be selected from the group consisting of Bacillus, Closridium, Cornebacterial, Lactobacillus, Lactococcus, Oenococcus, Streptococcus and Eubacterial cells. Various thermophilic bacterial cells, such as Thermoanaerobes (e.g., Thermoanaerobacterium saccharolyticum) can also be manipulated to increase furfural resistance and/or 5-HMF resistance as disclosed herein. Other thermophilic microorganisms include, but are not limited to, Bacillus spp., e.g., Bacillus coagulans strains, Bacillus licheniformis strains, Bacillus subtilis strains, Bacillus amyloliquifaciens strains, Bacillus megaterium strains, Bacillus macerans strains. Paenibacillus spp. strains or Geobacillus spp. such as Geobacillus stearothermophilus strains can be genetically modified. Other Bacillus strain can be obtained from culture collections such as ATCC (American Type Culture Collection) and modified as described herein.

Other embodiments provide for a yeast cell or fungal cell having increased expression of ucpA and fucO in combination with the deletion of the gene encoding yqhD or chromosomal integration of a gene encoding pntAB behind the adhE promoter (adhE::pntAB) and that exhibits improved ability to produce a desired product in the presence of furfural and 5-HMF as compared to a reference cell (e.g., a reference bacterial, yeast or fungal cell). In various embodiments, the bacterial, fungal or yeast cell contains a genetic construct comprising ucpA and fucO operably linked to a promoter comprising SEQ ID NO: 1. The yeast cell may be a Candida. Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

In other embodiments, the genetic modifications disclosed herein may be made to a fungal cell. “Fungi” as used herein includes the phyla Ascomycota, Basidiomycota, Chytridiomycota, and Zygomycota, Oomycota and all mitosporic fungi. A fungal cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

The fungal host cell may be a filamentous fungal cell. “Filamentous fungi” include all filamentous forms of the subdivision Eumycola and Oomycola (as defined by Hawksworth et al., Ainsworth and Bisby's Dictionary of the Fungi, 8th edition, 1995, CAB International, University Press, Cambridge, UK). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Miyceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press. Inc., New York; Ito et al., 1983, J. Bacteriol. 153: 163; and Hinnen et al., 1978, Proc. Natl. Acad. Sci. USA 75: 1920.

In various embodiments within this aspect of the invention, the bacterial cells can be Escherichia coli or Klebsiella oxytoca that have, optionally, been genetically modified to produce a desired product. In these embodiments, an isolated or recombinant bacterial cell is modified as disclosed herein to provide increased tolerance to furfural.

Various other aspects of the invention provide methods of producing ethanol, lactic acid, succinic acid, malic acid, acetic acid, 1,3-propanediol, 2,3-propanediol, 1,4-butanediol, 2,3-butanediol, butanol, pyruvate, dicarboxylic acids, adipic acid or amino acids. In these aspects of the invention, known bacterial, fungal or yeast cells that produce ethanol, lactic acid, succinic acid, malic acid, acetic acid, 1,3-propanediol, 2,3-propanediol, 1,4-butanediol, 2,3-butanediol, pyruvate, dicarboxylic acids, adipic acid or amino acids are manipulated in a manner that results in an increase in furfural tolerance for the bacterial, fungal or yeast cell (as compared to a reference bacterial, fungal or yeast cell). In various embodiments, the methods comprise culturing a bacterial, fungal or yeast cell producing a desired product (e.g., ethanol, lactic acid, succinic acid, malic acid, acetic acid, 1,3-propanediol, 2,3-propanediol, 1,4-butanediol, 2,3-butanediol, pyruvate, dicarboxylic acids, adipic acid or amino acids) and having increased UcpA activity, as compared to a reference cell, under conditions that allow for the production of the desired product. The desired product (e.g., ethanol, lactic acid, succinic acid, malic acid, acetic acid, 1,3-propanediol, 2,3-propanediol, 1,4-butanediol, 2,3-butanediol, pyruvate, dicarboxylic acids, adipic acid or amino acids) can, optionally, be purified from the culture medium in which the bacterial, fungal or yeast cell was cultured. In various other embodiments, the bacterial, fungal or yeast cells can be cultured in the presence of a hemicellulose hydrolysate.

As used herein. “isolated” refers to bacterial, fungal or yeast cells partially or completely free from contamination by other bacteria. An isolated bacterial, fungal or yeast cell (bacterial, fungal or yeast cell) can exist in the presence of a small fraction of other bacteria which do not interfere with the properties and function of the isolated bacterial, fungal or yeast cell (e.g., a bacterial, fungal or yeast cell having increased furfural tolerance). An isolated bacterial, fungal or yeast cell will generally be at least 30%, 40%, 50%, 60%, 70%, 80%, 85%, 90%, 95%, 98%, or 99% pure. Preferably, an isolated bacterial, fungal or yeast cell according to the invention will be at least 98% or at least 99% pure.

A “recombinant cell” is a bacterial, fungal or yeast cell that contains a heterologous polynucleotide sequence, or that has been treated such that a native polynucleotide sequence has been mutated or deleted. A “mutant” bacterial, fungal or yeast cell is a cell that is not identical to a reference bacterial, fungal or yeast cell, as defined herein below.

A wild-type bacterial, fungal or yeast cell is the typical form of an organism or strain, for example a bacterial cell, as it occurs in nature, in the absence of mutations. Wild-type refers to the most common phenotype in the natural population. “Parental bacterial, fungal or yeast strain”, “parental bacterial strain”, “parental fungal strain” or “parental yeast strain” is the standard of reference for the genotype and phenotype of a given bacterial, fungal or yeast cell and may be referred to as a “reference strain” or “reference bacterial, fungal or yeast cell”. A “parental bacterial, fungal or yeast strain” may have been genetically manipulated or be a “wild-type” bacterial cell depending on the context in which the term is used.

The terms “increasing”, “increase”, “increased” or “increases” refers to increasing by at least 5%, for example, 5, 6, 7, 8, 9, 10, 15, 20, 25, 30, 35, 40, 45, 50, 55, 60, 65, 70, 75, 80, 85, 90, 95, 99, 100% or more, a particular activity (e.g., increased UcpA activity). The terms “decreasing”, “decrease”, “decreased” or “decreases” refers to reducing by at least 5%, for example, 5, 6, 7, 8, 9, 10, 15, 20, 25, 30, 35, 40, 45, 50, 55, 60, 65, 70, 75, 80, 85, 90, 95, 99, 100% or more, a particular activity (e.g., any decreased activity). An increase (or decrease) in activity includes an increase (or decrease) in the rate and/or the level of a particular activity (e.g., furfural tolerance). “Growth” means an increase, as defined herein, in the number or mass of a bacterial, fungal or yeast cell over time.

The nucleic and amino acid sequence of the ucpA gene (SEQ ID NO: 4) and polypeptide (UcpA; SEQ ID NO: 5) are known in the art (see, for example, EMBL-Bank Accession No. X99908.1 which is hereby incorporated in its entirety and are provided in the sequence listing appended hereto). Likewise, the nucleic acid and polypeptide sequences for FucO are also known in the art. The nucleic and amino acid sequence of the FucO gene (SEQ ID NO: 6) and polypeptide (SEQ ID NO: 7) are known in the art (see GenBank Accession Nos. ADT76407.1, for example and GenBank Accession No. CP002185, REGION: 3085103-3086251, VERSION CP002185.1 GI:315059226, Archer et al., BMC Genomics 12 (1), 9 (2011), each of which is hereby incorporated by reference in its entirety) and are provided in the sequence listing appended hereto.

In one aspect of the invention, bacterial cells having increased UcpA and FucO activity can also have the activity of YqhD decreased or altered, as compared to the activity of YqhD in a reference bacterial cell. Activity is decreased or altered by methods known in the art, including but not limited to modification of yqhD (e.g. by inserting, substituting or removing nucleotides in the gene sequence or complete chromosomal deletion of the gene). Thus, this aspect of the invention can also provide a bacterial cell wherein expression of UcpA and FucO is increased, as compared to a reference bacterial cell and expression of the yqhD is decreased as compared to the expression of yqhD in a reference bacterial cell. Methods for altering the activity of YqhD and inactivating yqhD are known in the art, see for example PCT/US2010/020051 (PCT publication WO 2010101665 A1) which is hereby incorporated by reference in its entirety.

The invention provides for a bacterial, fungal or yeast cell that has an increased resistance to furfural, increased expression of FucO and UcpA protein or mRNA and in combination with the deletion of the gene encoding yqhD or chromosomal integration of a gene encoding pntAB behind the adhE promoter (adhE::pntAB) and that exhibits improved ability to produce a desired product in the presence of furfural and 5-HMF as compared to a reference cell (e.g., a reference bacterial, yeast or fungal cell). In various embodiments, the bacterial, fungal or yeast cell contains a genetic construct comprising ucpA and fucO operably linked to a promoter comprising SEQ ID NO: 1 and which, as compared to a reference bacterial, fungal or yeast cell, exhibits at least one of: 1) increased growth in the presence or absence of furfural as compared to a reference bacterial, fungal or yeast cell; 2) increased growth and increased production of a desired product as compared to a reference bacterial, fungal or yeast cell; 3) increased growth and increased production of a desired product, in the presence of furfural, as compared to a reference bacterial, fungal or yeast cell; 4) increased growth in the presence of a hydrolysate as compared to a reference bacterial, fungal or yeast cell; and 5) increased production of a desired product as compared to a reference bacterial, fungal or yeast cell.

Various aspects of the invention provide for the use of a variety of hydrolysates for the production of a desired product, including but not limited to, hydrolysate derived from a biomass, a hemicellulosic biomass, a lignocellulosic biomass or a cellulosic biomass. Yet other aspects of the invention provide a bacterial, fungal or yeast cell with increased resistance to furfural, wherein the bacterial, fungal or yeast cell is capable of producing a desired product as a primary fermentation product, wherein optionally, the primary fermentation product is produced under anaerobic or microaerobic conditions.

The invention also provides for a method for producing a desired product from a biomass, a hemicellulosic biomass, a lignocellulosic biomass, a cellulosic biomass or an oligosaccharide source comprising contacting the biomass, hemicellulosic biomass, lignocellulosic biomass, cellulosic biomass or oligosaccharide with any of the isolated or recombinant bacterial, fungal or yeast cell of the invention thereby producing the desired product from a biomass, hemicellulosic biomass, lignocellulosic biomass, cellulosic biomass or an oligosaccharide source.

Further, the invention provides for a method for producing a desired product from a biomass, a hemicellulosic biomass, a lignocellulosic biomass, a cellulosic biomass or an oligosaccharide source in the presence of furfural comprising contacting the biomass, hemicellulosic biomass, lignocellulosic biomass, cellulosic biomass or oligosaccharide with the isolated or recombinant bacterial, fungal or yeast cell of the invention, thereby producing the desired product from a biomass, hemicellulosic biomass, lignocellulosic biomass, cellulosic biomass or an oligosaccharide source.

Other aspects of the invention provide for isolated polynucleotides and isolated polypeptides comprising any one of SEQ ID NOs: 1-3 and 13 or 14. In particular embodiments, any one of SEQ ID NOs: 1-3 can be operably linked to a heterologous polynucleotide sequence (i.e., a gene other than yadC) in order to facilitate expression of the heterologous sequence within a host cell. Various other embodiments include vectors comprising any one of SEQ ID NOs: 1-3 operably linked to a heterologous polynucleotide sequence or vectors comprising SEQ ID NO: 13 or 14. Host cells comprising such vectors are another aspect of the disclosed invention.

Following are examples which illustrate procedures for practicing the invention. These examples should not be construed as limiting. All percentages are by weight and all solvent mixture proportions are by volume unless otherwise noted. It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### Materials and Methods

### Methods for Gene Deletion and Integration

The methods of seamless chromosomal deletion, gene replacement, and integration were previously described using Red recombinase technology (27). In general, primers “up” and “down” were used to amplify target genes and adjacent regions (200-400 bp upstream and downstream to ORF). Resulting PCR products were cloned into the pCR2.1 TOPO vector. Primers with the designation “1” and “2” (“10” and “20” in some cases) were used to amplify the backbone of the plasmid by inside-out PCR, omitting the coding region of target gene. The PCR fragments were ligated to cat-sacB cassette (amplified from pLOI4162) to create the template for integration (1). After removal of cat-sacB, the self-ligated plasmid contains only the adjacent regions of target region allowing a seamless deletion (27). Plasmids and primers used in strain constructions are listed in Table 1.

Constructions of Plasmids for fucO-ucpA Expression and Chromosomal Integration

pLOI5229 (pTrc fucO-ucpA)

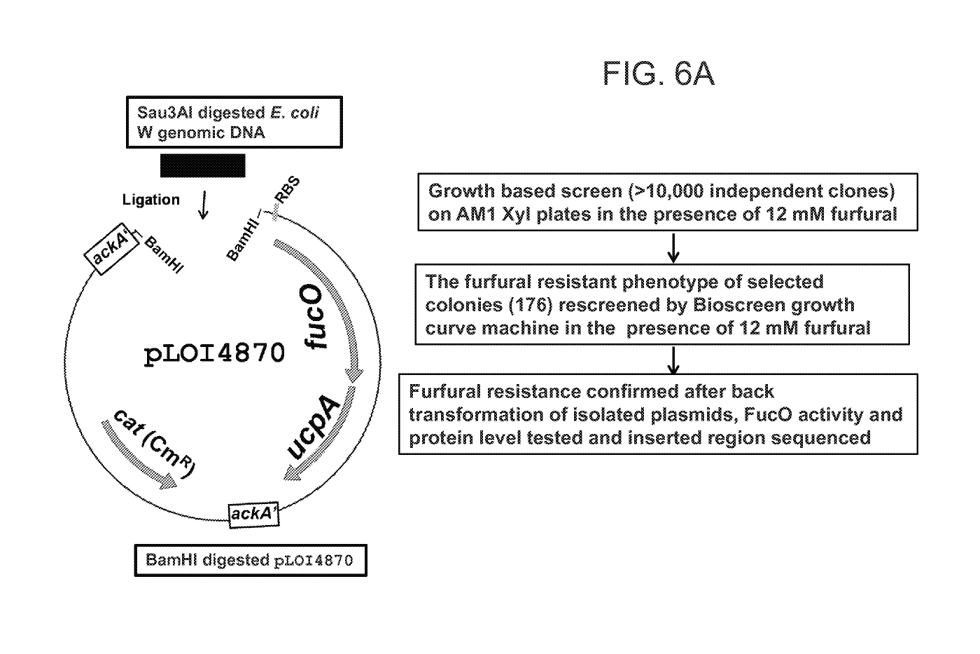

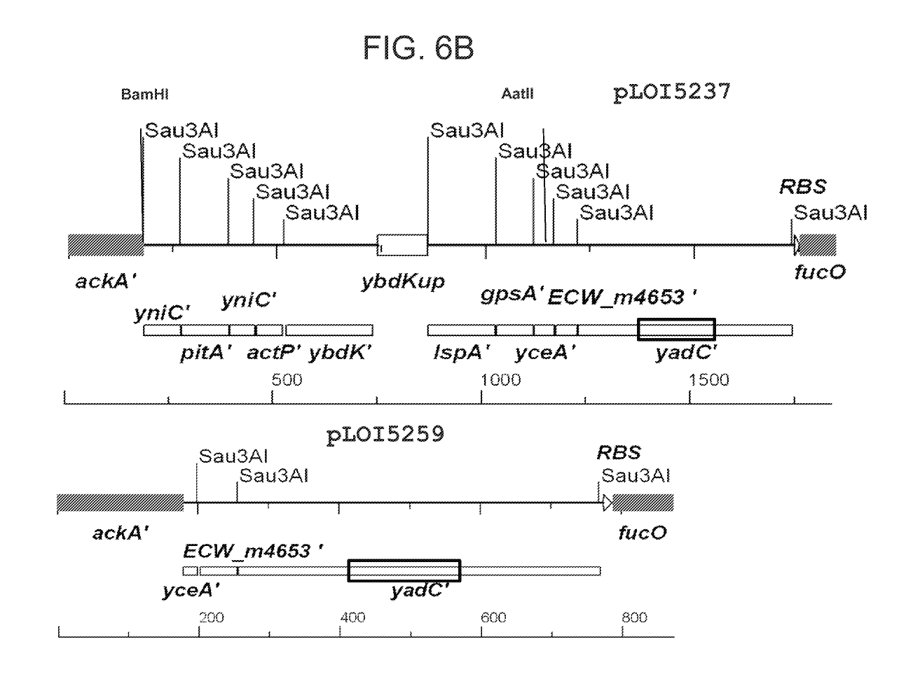

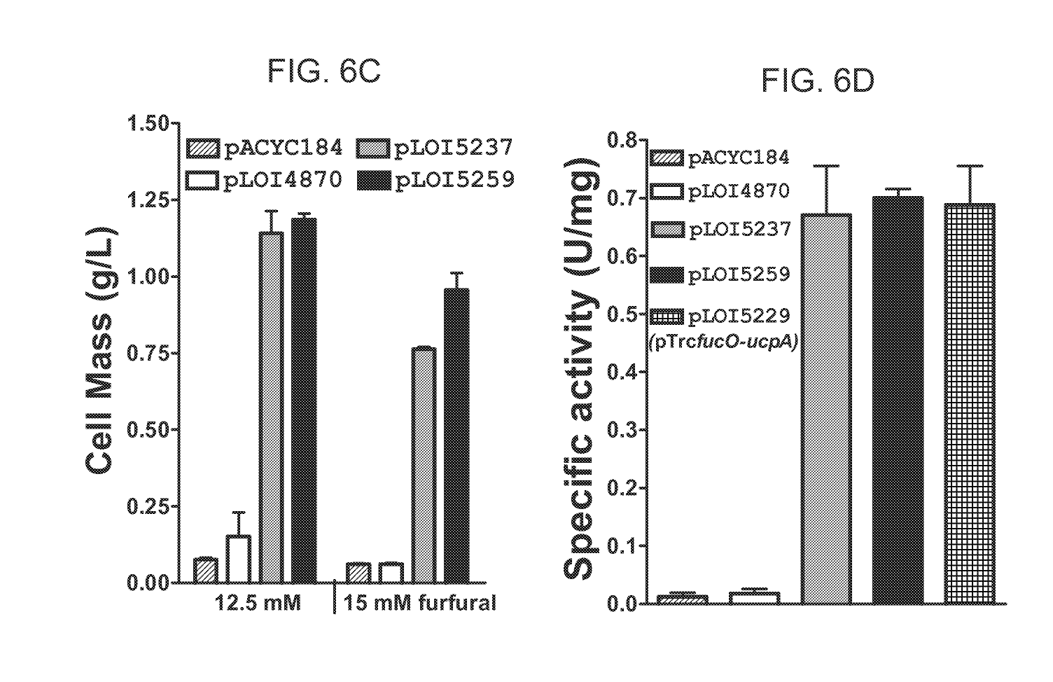

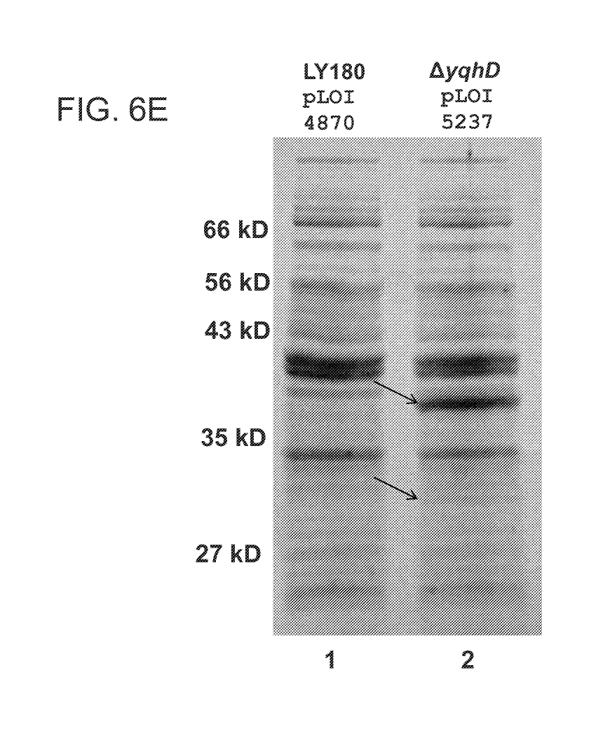

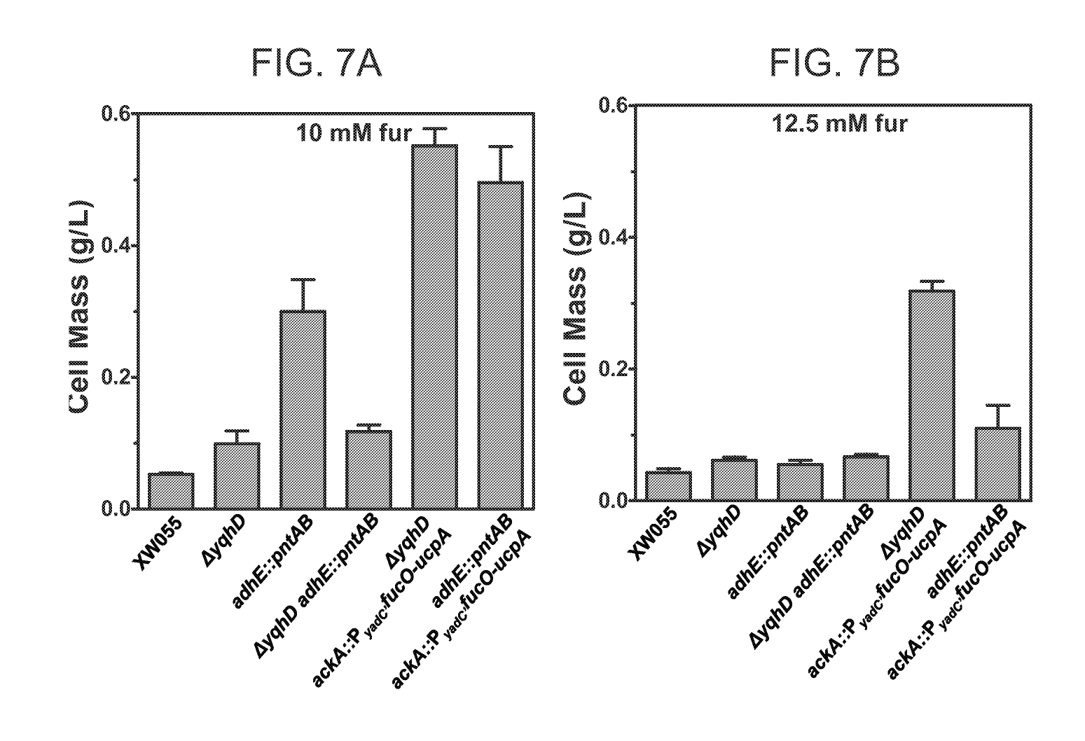

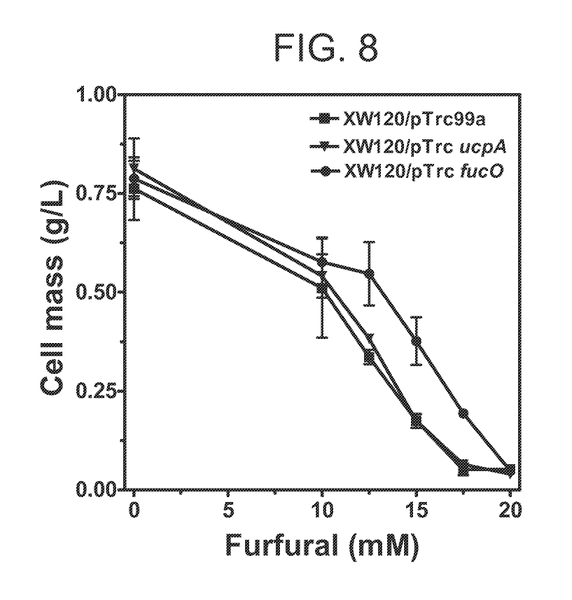

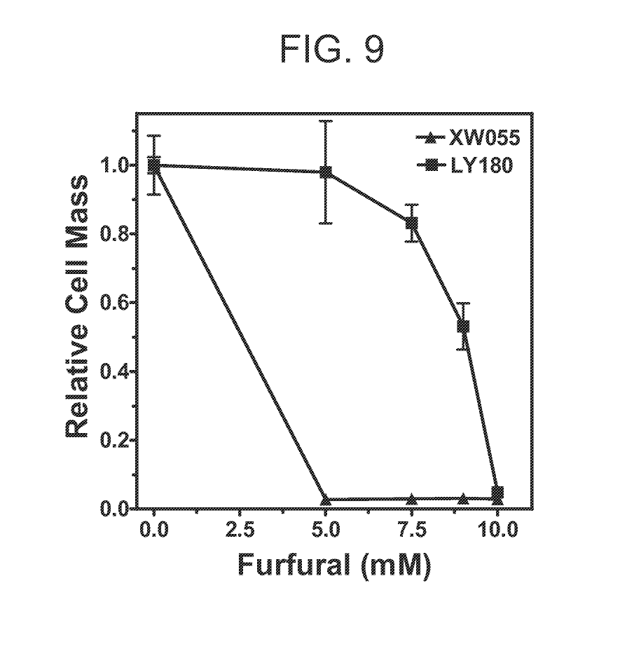

The DNA sequence of fucO (ribosome binding site, coding region and terminator) was previously cloned into pTrc99a (pLOI4319) (17). The whole plasmid of pLOI4319 (17) was amplified by PCR using primers pTrcFucO-UcpA left and pTrcFucO-UcpA right to open the plasmid precisely after fucO stop codon and to create the fragment containing the plasmid backbone and fucO ORF. The fragment containing intergenic sequence (AATTGAAGAAGGAATAAGGT; SEQ ID NO: 15) and ucpA ORF was assembled by PCR using E. coli genomic DNA as template and primers pTrcFucO-UcpAORFup and pTrcFucO-UcpAORFdown. Both PCR fragments contain a more than 50 bp identical sequence at each end provided by primers. The two pieces of DNA were joined by CloneEZ® PCR Cloning Kit from GenScript (Piscataway, N.J.) to produce pLOI5229. The protein level of FucO produced from pLOI5229 is equal to that from pLOI4319 (approximately 0.7 U/mg protein) (FIG. 6D) (17).

pLOI4857 (Cloning Wild-Type ackA and its Adjacent Region into pACYC184) The fragment of E. coli ackA ORF and its adjacent region (200 bp upstream and downstream from coding region) was amplified by PCR using primers ackAup200 and ackAdown200. Using primers pACYC-up and pACYC-down, the plasmid backbone of pACYC184 excluding let ORF (1.2 kb) was also amplified. After phosphorylation, these two DNA fragments were ligated to form plasmid pLOI4857.

pLOI4859 (Replacing ackA ORF with fucO-ucpA to Create ackA::fucO-ucpA Cassette)

Primers ackA 1 and ackA 2 were used to amplify the sequence from pLOI4857 precisely excluding the ackA ORF by PCR. Primers ackApAC up and ackApAC down were used to amplify the fucO-ucpA fragment from pLOI5229. The two pieces of DNA were joined by CloneEZ® PCR Cloning Kit, designated pLOI4859.

pLOI14869 (Reducing the Size of pLOI4859)

Primers pACY PacI and pACY HindIII were used to amplify the backbone of pACYC184 omitting let and downstream sequence (1.9 kb). PacI and HindIII sites in primers were added to the two ends of the PCR fragment. Primers HindIII ackA fucO and ackA fucO PacI were used to amplify the fucO-ucpA cassette with flanking ackA′ regions using pLOI4859 as a template. These primers included PacI and HindIII sites at the ends. These two PCR products ligated to create plasmid pLOI4869.

pLOI4870 (Adding Unique BamHI Site and Ribosomal Binding Region)

The full length of plasmid pLOI4869 was amplified by inside-out PCR using primers fucO RBS and fucO BamHI. After phosphorylation and self-ligation, the resulting plasmid was designated pLOI4870. This plasmid contained a promoter-probe cassette consisting of a unique BamHI site for ligation of Sau3A1 fragments followed by an adhE ribosomal binding site, fucO ORF, an intergenic sequence and ucpA ORF (FIG. 6). This cassette is bordered by sequence homologous to upstream (omitting part of ackA native promoter and ribosomal binding site) and downstream sequences to ackA ORF that can be used to guide chromosomal integration (FIG. 6).

Growth-Based Screen for Surrogate Promoters to Express the fucO-ucpA Cassette

E. coli genomic DNA was completely digested with Sau3AI and ligated into BamHI-treated pLOI4870 to create a plasmid library containing varied sequences between ackA upstream sequences (ackA′) and the ribosomal binding site of fucO (FIG. 6A). More than 10,000 colonies were pooled and used to prepare a master library of plasmid DNA. The plasmid library of surrogate promoters was transformed into XW092 (LY180 ΔyqhD) with selection on AM1-xylose plates containing 12 mM furfural and 40 mg/L chloramphenicol. Plates were incubated under argon. Large colonies (176 clones) were isolated from more than 10,000 transformants. These were further screened using a BioScreen C growth curve analyzer (Piscataway, N.J.). Control strains XW092(pACYC184), XW092(pLOI4870) and clones with a large colony phenotype were inoculated in a 100-well honeycomb plate containing 400 μl of AM1 xylose medium with 40 mg/L chloramphenicol. Optical density was measured at 30-min intervals with 10 s shaking immediately before each reading. After incubation for 16 h, these seed cultures were diluted to an initial optical density of 0.1 and inoculated again in AM1 media containing 12 mM furfural and 40 mg/L chloramphenicol. Growth curves were monitored. The single clone with the highest furfural resistance was selected and designated pLOI5237 (FIGS. 6B and 6C). XW092(pLOI5237) also showed much stronger NADH-linked furfural reductase activities (approximately 0.7 U/mg protein) (FIG. 6D) and the enhanced putative FucO and UcpA bands (FIG. 6E) compared to XW092(pLOI4870).

The promoter fragment in pLOI5237 (1.6 kb) was composed of 10 independent Sau3AI fragments (FIG. 6B), each from a different region of the E. coli genome. It does not have any known promoter and any complete gene. Approximately 1 kb of upstream sequence containing 8 of these fragments was deleted by digestion with BamH1-AatII (self-ligation to create pLOI5259) (FIG. 6B), with no decline in furfural tolerance (FIG. 6C) or furfural reductase activity (FIG. 6D). Analysis of this sequence with the web-based program Neural Network Promoter Prediction 2.2 (http://www.fruitfly.org/seq_tools/promoter.html) and BPROM (http://linux1.softberry.com/berry.phtml) both predicted a promoter in an internal segment of the yadC coding region near the center of this fragment (FIG. 6B).

Sequences of Promoter Fragments from pLOI15237 and pLOI5259 (Subclone)

The predicted promoter region (BPROM and Neural Network Promoter Prediction) is underlined and bold. The sequence of ackA′ upstream and partial fucO ORF (downstream) are italicized and underlined.

Promoter fragment (1.6 kb) from pLOI5237
TACTTGAGTCGTCAAATTCATATACATTATGCCATTGGCTGAAAATTACGCAAAATGGCA
TAGACTCAAGATATTTCTTCCATCATGCAAAAAAAATTTGCAGTGCATGATGTTAATCAT
AAATGTCGGTGTCATCATGCGCTACGCTCTATGGCTCCCTGACGTTTTTTTAGCCAGG
(ackA′ upstream sequence)
(SEQ ID NO: 16)
ATCCACGTTTTGTATTAGCAAACGTCAAACTCTCATCGCTGCAAATCACCGCAAA
AGACCTTCTCGGTTAATGACCAGGGGCAGTGATCGTCTCATGGCCTTGCCATGGT
GTTCTCTATGTTGCTGGCGGCGATTATCTGGAACCTGGGTACCTGGTACTTTGGTT
TACCTGCATCCAGCTCTCATACGCTGATTGGCGCGATCGCTTCTAAAGCAGCCCG
CATGCGTTCCATCGTCGTTCCTGCGCCAGAAGCGCAAAATGATCGGCCAGCCCAC
CAGGAAGCCCAGCGAGTAAATTAAGCCGTCATAGCCGGAGGTAAACACCAGCGC
GGAGATCTCATTTTTAACCGCGTCAATCAGCATTGAAGAGTCCTGGCTTAAGTCA
TAGCCCGGCGGATTAACCACCTGCATTTCCAGTTCAATACCGAGGGTAAAAGGTT
CAGAAACATGAAAATCGGGTAATGGCATAGGTTTCTCTTAAGTTGGCGTTTTCTA
TTCAGTATAGAAGTCGGAGCGGCTGGGCGAGATGCGGAAGTTCTGGAATGTTTCT
TTTTTTGGTGATGGTGACTGAAGCAATTTGGCTACTTTTGCAATGTGACAAGTTAT
GGCACGGCTGGCTGGTGGCGAAGAATTTTGACGATTGAGGCATGCAGAAAAAAA
ACGGGTTCAGCTTTCAGTTGATCCTCCCAGAACTTTGCTCTGGGGGGATACGGTC
CCCGCTGTTCCCCGTCGCTTAATCTGCATTATGCCGCGTAACTATGGCGCGGCGTT
TAAGTTTCCTTGCCGATAGCGGCGGCTGGCAGCGTTGGTTCTTTGCCGGTATTGC
GATTGGTATTAGCGTGATCAAATTCCGCTGGCGGTTATCTCTGGCCCAACGTTTG
CGAAAGAACTGGCGGCAGGTTTACCTACAGCTATTTCGCTGGCCTCGACCGATCA
GGAATGCCCAGTGTTGTATTCAGACGTCCACGTGACTTATTAAAGATCTTTACTG
CGGCTATACTCCTGCGACGCTAATTGAGCAGCTTTTTGGTAAGATTGATCAAAAA
TGGAGAGAAACGGGGCCGAATGGCGATGCTACTGTCATATTCAGATATGCAACA
AGTACAAATAATTTAGTTTTCTACAAACCGACGCAGCTTGGACCTACAGGTGTAA
AATTACAGTGGAGTCAGTTAGATACCGCTTCTGGTGGTGGTTTTCTTTATTGCAAC
AGAAGTGATAGCACAAGTGGTAGCGCAATGCGTATTGAAAATGCAATGGTTGA
CTCAGGTAAAATGTATGGCTCCCATAAATTATTTAATACATCAGTTCCTGGT
TTGTATTACACATTATTAATTTCAAACATGTGGTCAGCTTACGGTACCGTAACTA
ACGTTAGTTCACCTGGGATATACATTGGTGACTCTGCAGAACAATATTTTTCGTG
GTATAATCCAAGCGAAGACGTGTTATATTGGAGTTGCAATAATGCGAATAGCAC
CCGTAAATACTGGGCTGTAGGTGGTATTTATCAGACCCTTACAATTGAATTCTAT
ACAGATACAAACTTTGATCCATATCAGGAGAGCATTATGATGGCTAACAGAATGAT
TCTGAACG . . . (fucO downstream ORF)
Subcloned promoter fragment (0.6 kb) from pLOI5259
(SEQ ID NO: 17)
GTCGTCAAATTCATATACATTATGCCATTGGCTGAAAATTACGCAAAATGGCATAGACTC
AAGATATTTCTTCCATCATGCAAAAAAAATTTGCAGTGCATGATGTTAATCATAAATGTC
GGTGTCATCATGCGCTACGCTCTATGGCTCCCTGACGTTTTTTTAGCCAGG(ackA′
upstream sequenee)ACCACGTGACTTATTAAAGATCTTTACTGCGGCTATACTCCTG
CGACGCTAATTGAGCAGCTTTTTGGTAAGATTGATCAAAAATGGAGAGAAACGG
GGCCGAATGGCGATGCTACTGTCATATTCAGATATGCAACAAGTACAAATAATTT
AGTTTTCTACAAACCGACGCAGCTTGGACCTACAGGTGTAAAATTACAGTGGAGT
CAGTTAGATACCGCTTCTGGTGGTGGTTTTCTTTATTGCAACAGAAGTGATAGCA
CAAGTGGTAGCGCAATGCGTATTGAAAATGCAATGGTTGACTCACGTAAAATG
TATGGCTCCCATAAATTATTTAATACATCAGTTCCTGGTTTGTATTACACATT
ATTAATTTCAAACATGTGGTCAGCTTACGGTACCGTAACTAACGTTAGTTCACCT
GGGATATACATTGGTGACTCTGCAGAACAATATTTTTCGTGGTATAATCCAAGCG
AAGACGTGTTATATTGGAGTTGCAATAATGCGAATAGCACCCGTAAATACTGGG
CTGTAGGTGGTATTTATCAGACCCTTACAATTGAATTCTATACAGATACAAACTT
TGATCCATATCAGGAGAGCATTATGATGGCTAACAGAATGATTCTGAACG . . . (fucO
ORF).

### Results

### Epistatic Interactions Among Four Furfural Resistance Traits in Ethanologenic LY180

Previous studies have shown that deletion of yqhD and increased expression of fucO, ucpA, or pntAB from plasmids each improved growth of ethanologenic E. coli LY180 in the presence of 10 mM furfural (17-19, 22). Further constructions (see Table 1) were made to allow a comparison of all combinations of these genetic traits using pTrc99a-based plasmids for expression of target genes (fucO, ucpA, and fucO-ucpA). Three new derivatives of LY180 were constructed for use as host strains: ΔyqhD, adhE::pntAB and ΔyqhD adhE::pntAB. Integration of pntAB behind the adhE promoter in LY180 provided furfural tolerance equivalent to pTrc99a expressing pntAB (uninduced). Higher levels of pntAB expression with inducer were inhibitory in the absence or presence of furfural (18).

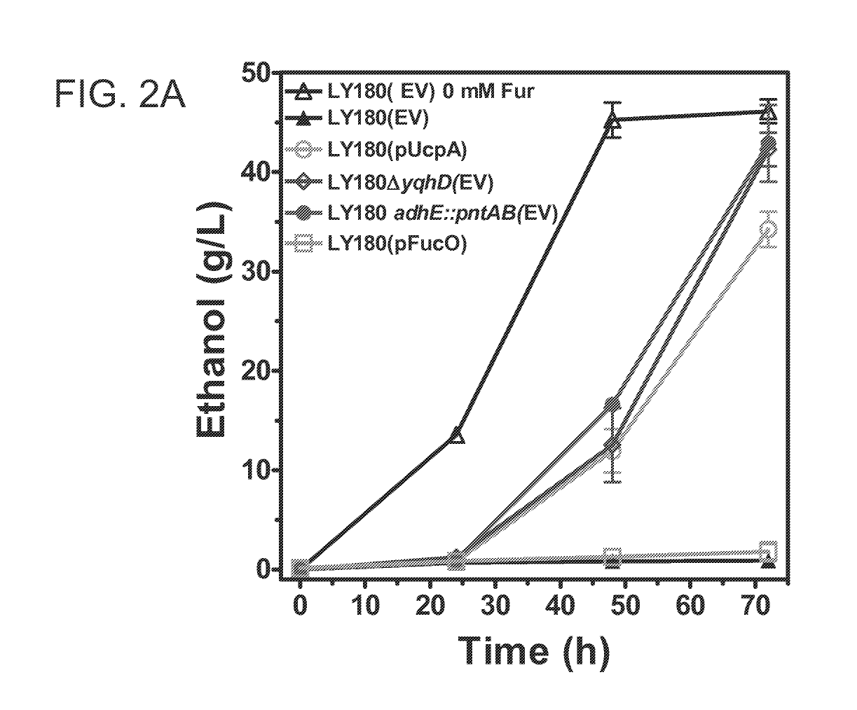

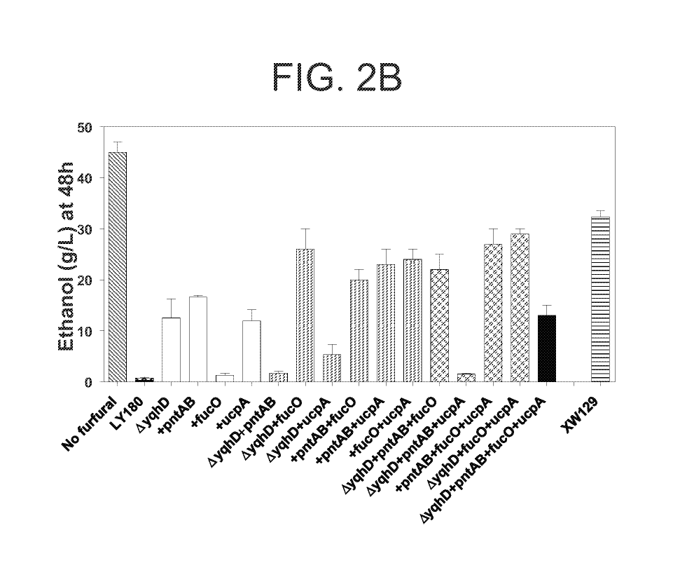

Ethanol production from 100 g/L xylose was complete after 48 h in control cultures lacking furfural (FIG. 2A). Ethanol production at this time point was selected as a comparative measure of tolerance to 15 mM furfural. All individual traits except fucO improved ethanol production in the presence of 15 mM furfural (FIG. 2A). Combinations of two traits (FIG. 2B) were more effective than single traits with two exceptions: 1) ΔyqhD with pntAB integration and 2) ΔyqhD with the ucpA plasmid (pLOI4856). All binary combinations with fucO were beneficial. Since growth and ethanol production were also inhibited by excess pntAB expression (18), the negative interactions between pntAB (increased NADPH production) and ΔyqhD (reduced NADPH consumption) could result from a similar problem. The poor performance of LY180 ΔyqhD containing the ucpA plasmid suggests that this cryptic gene may be associated with a similar action. Among ternary combinations, the combination of ΔyqhD adhE::pntAB and ucpA plasmid was particularly sensitive to furfural inhibition. Ethanol titer was low (13 g/L) when all four genetic traits were combined, comparable to strains with a single resistance trait (FIG. 2B). The most effective combinations were plasmid expression of fucO-ucpA in a host strain with either ΔyqhD or adhE::pntAB. Both constructs produced close to 30 g/L ethanol after 48 h in medium with 15 mM furfural, about 70% of the ethanol titer in control fermentations without furfural (FIG. 2B).

SEQ ID NO. 13: nucleic acid sequence for adhE::pntAB (adhE open reading frame is replaced by pntAB open reading frame: bold and italic):

GACAGCATTTTTCACCTCCTAACTACTTAAAATTGCTATCATTCGTTATTGTTATCTAGTTG
TGCAAAACATGCTAATGTAGCCACCAAATCATACTACAATTTATTAACTGTTAGCTATAATG
GCGAAAAGCGATGCTGAAAGGTGTCAGCTTTGCAAAAATTTGATTTGGATCACGTAATTACT
ACCCAGAAGTGAGTAATCTTGCTTACGCCACCTGGAAGTGACGCATTAGAGATAATAACTCT
AATGTTTAAACTCTTTTAGTAAATCACAGTGAGTGTGAGCGCGAGTAAGCTTTTGATTTTCA
TAGGTTAAGCAAATCATCACCGCACTGACTATACTCTCGTATTCGAGCAGATGATTTACTAA
AAAAGTTTAACATTATCAGGAGAGCATT

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

TCAGTAGCGCTGTCTGGCAACATAAACGGCCCCTTCTGGGCAATGCCGATCAGTTAAGGATT
AGTTGACCGATCCTTAAACTGAGGCACTATAACGGCTTCCACAACAGGGAGCCGTTTTCTTA
TGCCACTTCTCAATGATCTGCTCGATTTCAGTGACCATCCGCTTATGCCTCCGCCCTCTGCA
CAACTATTTGCAGAACACCTTCCCACCGAGTGGATACAACACTGCCTGACGCTTTCTGCTCA
TGCGACCGTTCGCCGCCGTCGTTTACCGGGGGACATGGTTATCTGGATGGTGGTGCAATGAG
CCAATTACCGATGTTGTTCGCCGTCTGAACCTGAGCGCGGATGGCGAAGCGGGGATGAACCT
GCTGGCCCGCAGCGCTGTCACCCAGGCG

Constructing Plasmid-Free Strains for Ethanol Production (Integration of fucO-ucpA)

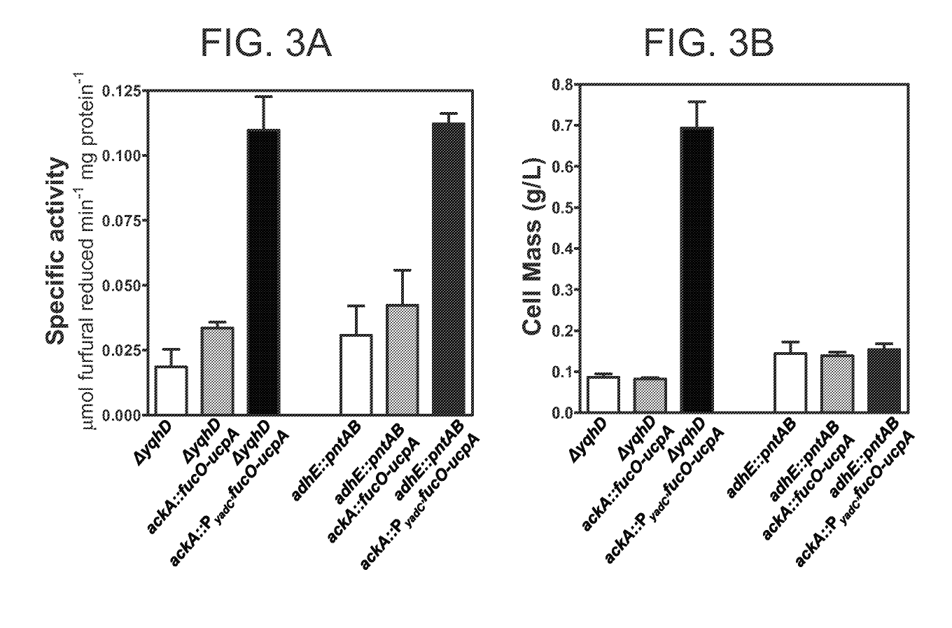

The use of plasmids, antibiotics, and expensive inducers allowed an investigation of gene interactions but is unlikely to provide the desired genetic stability needed for commercial strains. Chromosomal integration of fucO-ucpA behind a strong promoter such as ackA (highly expressed in mRNA arrays) (18, 20, 22) was tested as a replacement for plasmid pTrc fucO-ucpA in LY180 adhE::pntAB and LY180 ΔyqhD. However, FucO activity of the integrated strains was low (FIG. 3A) and furfural tolerance (12.5 mM) was unchanged (FIG. 3B). Integration behind the strong pflB promoter (18, 20, 22, 23) also did not provide sufficient expression of fucO-ucpA for furfural tolerance. Clearly, a more efficient approach was needed.

A function-based selection was used to identify a useful promoter. A promoter probe vector was constructed for fucO-ucpA as a derivative of pACYC184 (low copy) with an appropriately engineered upstream BamH1 site (FIG. 6A). Random Sau3A1 fragments (E. coli W chromosome) were ligated into this site and resulting plasmids transformed into LY180 ΔyqhD. After selection for large colonies on furfural (12 mM) plates and further screening, the most effective promoter was identified by sequencing as a 600 bp internal fragment of the E. coli yadC gene, designated P_{yadC′} in plasmid pLOI5259 (FIG. 6B). With this promoter, constitutive expression of fucO on a low copy plasmid (pACYC184) was equal to induced expression of fucO from a high copy plasmid (pTrc99a) (FIG. 6).

The expression cassette from pLOI5259 (ackA′::P_{yadC′}fucO-ucpA-ackA′) was amplified by PCR (Table 1) and integrated into the chromosomes of LY180 ΔyqhD and LY80 adhE::pntAB by precisely replacing the ackA coding region including 22 bp immediately upstream. Resulting strains were designated XW129 and XW131, respectively. Although both integrated strains produced 4-fold to 6-fold higher FucO activity than the respective parent strain (FIG. 3A), furfural tolerance was only improved in XW129 (FIG. 3B). It is possible that the higher level of FucO produced with plasmids (0.7 U/mg protein; FIG. 6D) is required to increase tolerance in the adhE::pntAB strain (XW131) where yqhD remains functional.

Integration of Traits Restored Ethanol Fermentation in 15 mM Furfural.

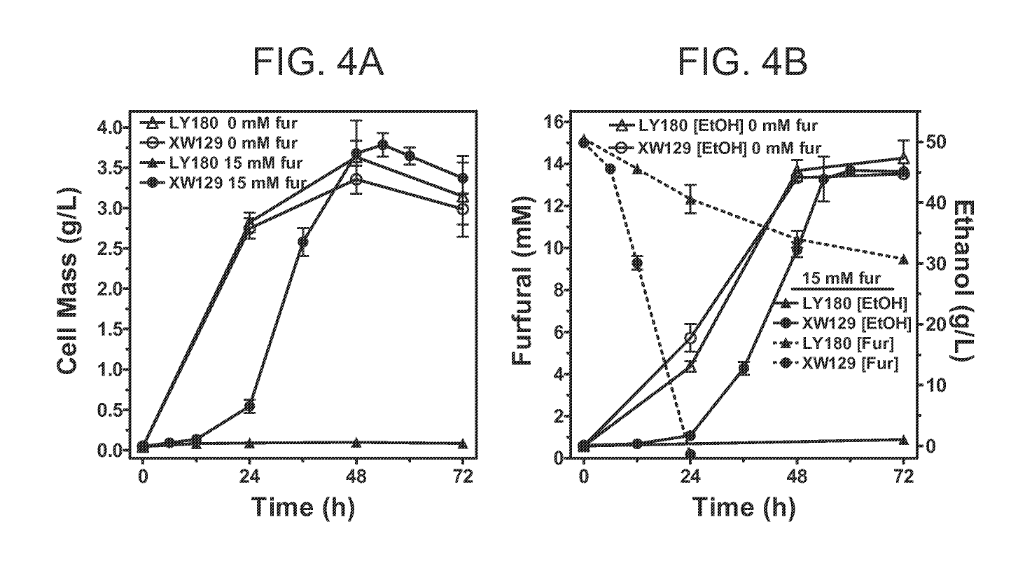

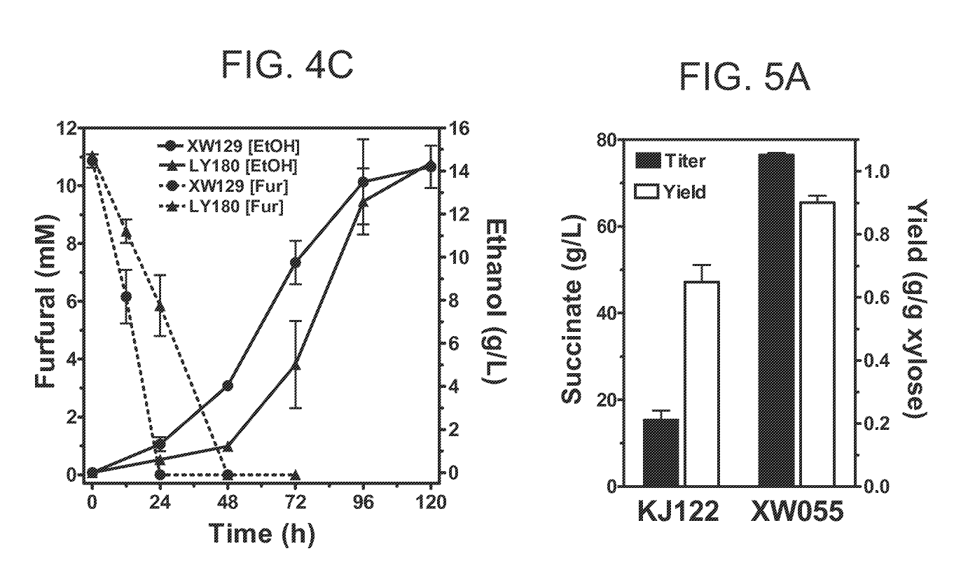

Strain XW129(LY180 ΔyqhD ackA::P_{yadC′}fucO-ucpA) was compared to the parent LY180 during batch fermentation in AM1 mineral salts medium (100 g/L xylose) with and without 15 mM furfural (FIGS. 4A and 4B). In the absence of furfural, ethanol yields for both strains were equal. In the presence of 15 mM furfural, growth and fermentation of LY180 was completely blocked. Only 5 mM furfural was metabolized (reduce to furfuryl alcohol) by LY180 after 72 h. Addition of 15 mM furfural delayed the growth of strain XW129 by 24 h, during which time furfural was fully reduced. However, the time required to complete fermentation was extended by only 6 h. The final ethanol yield for strain XW129 with 15 mM furfural was equal to the control without added furfural, 90% of the theoretical yield. Despite being 6-fold lower in FucO activity (FIG. 3A and FIG. 6D), ethanol titers (32 g/L after 48 h) for strain XW129 (LY180 ΔyqhD ackA::P_{yadC′}fucO-ucpA) with integrated fucO-ucpA were equivalent to LY180 ΔyqhD with induced expression of fucO-ucpA from plasmids (FIG. 2B). This suggests that the metabolic burden of plasmid maintenance and producing larger amounts of target protein (FucO, UcpA) may have countered any benefit from the additional activities.

Furfural-Resistance Traits Also Increased Resistance to Hemicellulose Hydrolylsate.

Furfural is regarded as one of the more important inhibitors in dilute acid hydrolysates of hemicellulose (6-8). This was confirmed in part by a comparison of batch fermentations containing sugarcane bagasse hemicellulose hydrolysate (FIG. 4C). The onset of rapid ethanol production was delayed in hydrolysate, similar to the delay with 15 mM furfural in AM1 medium containing 10% xylose (FIG. 4B). The onset of rapid ethanol production in AM1 medium with furfural and in hydrolysate medium (LY180 and XW129) again coincided with the depletion of furfural. Although total fermentation time in hydrolysate medium and final ethanol titers were similar for both the parent LY180 and the mutant XW129, the furfural-resistant mutant XW129 reduced furfural at twice the volumetric rate of LY180. This more rapid reduction of furfural by XL129 shortened the initial delay in ethanol production by 24 h, half that of the parent (FIG. 4C).

Re-Engineering E. coli KJ122 for Conversion of Hemicellulosic Hydrolysates to succinate

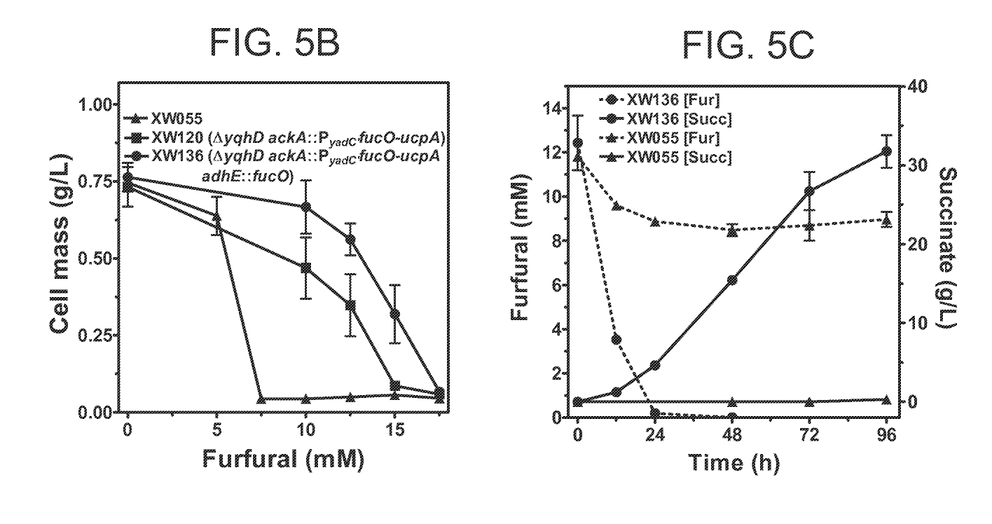

Strain LY180 is derived from E. coli KO11, a sequenced strain that has acquired many mutations during laboratory selections for growth in mixed sugars, high sugars, lactate resistance, and other conditions (24-26). It is possible that some of the mutations in KO11 or the heterologous genes encoding ethanol production in this strain may be critical for engineering furfural tolerance and improving resistance to hemicellulose hydrolysate. To address this concern, we have reconstructed the optimal traits for furfural-resistance in KJ122, a succinate-producing derivative of E. coli C (27). Initially, strain KJ122 was unable to effectively ferment 100 g/L xylose (FIG. 5A). Mutants with 5-fold improvement of succinate titer were readily selected after 40 generations of serial cultivation in xylose AM1 medium. A clone was isolated and designated XW055 with a succinate yield from xylose of 0.9 g/g, equivalent to the yield previously reported for glucose (27).

The same genetic tools used to construct furfural tolerance in ethanol-producing biocatalysts were used to engineer XW055 (FIG. 7 and FIG. 5B). As with ethanol biocatalysts, combining a yqhD deletion with integration of pntAB was not helpful (FIG. 7). The most effective combination for succinate production was ΔyqhD and ackA::P_{yadC′}fucO-ucpA, resulting in strain XW120 (FIG. 7 and FIG. 5B). These genetic changes increased the minimal inhibitory concentration of furfural from 7.5 mM (XW055) to 15 mM (XW120). Plasmid derivatives of pTrc99a expressing fucO alone and ucpA alone were tested in XW120. Addition of a fucO plasmid further increased furfural tolerance (FIG. 8). The benefit of this plasmid was supplied by another chromosomal integration, replacing the coding region of adhE with the coding region of fucO to make XW136. The additional expression of fucO from the adhE promoter increased furfural tolerance to 17.5 mM (FIG. 5B).

XW055 and the furfural-resistant mutant XW136 (XW055, ΔyqhD ackA::P_{yadC′}fucO-ucpA adhE::fucO) were compared during batch fermentation using hemicellulose hydrolysate as a source of sugar (FIG. 5C). Hydrolysate medium contained 12 mM furfural and completely inhibited growth and fermentation of the parent. During 96 h of incubation, the parent reduced only 3 mM furfural and was unable to grow or effectively ferment hemicellulose sugars. In contrast, furfural (12 mM) was completely reduced within 24 h by the furfural-resistant strain XW136. With this strain, fermentation of hemicellulose sugars (primarily xylose) into succinate was complete after 96 h with a yield of 0.9 g/g. This succinate yield from hemicellulose sugars was equivalent to that of the parent organism (KJ122) during the fermentation of glucose in AM1 mineral salts medium without furfural (27).

Discussion

Importance of Furfural as an Inhibitor in Hemicellulose Hydrolysate

Microbial biocatalysts can be used to produce renewable chemicals from lignocellulosic sugars. Large scale implementation of biobased processes has the potential to replace petroleum for solvents, plastics, and fuels without disrupting food supplies or animal feed. Costs for such processes remain a challenge and can be reduced by developing biocatalysts that are tailored for specific feedstocks. Inhibitors formed during the deconstruction of lignocellulose such as furfural are part of this challenge. Our studies demonstrate that removal of furfural is essential prior to rapid growth and metabolism of sugars by E. coli biocatalysts (FIG. 4B, FIG. 4C, and FIG. 5C).

Furfural, a natural product from the dehydration of pentose sugars (7, 8), serves as one of the barriers to effective fermentation of hemicellulose hydrolysates. Previous studies have shown that furfural was unique in binary combinations of inhibitors, increasing the toxicity of other compounds (soluble lignin products, formate, acetate, etc.) in hemicellulose hydrolysates (13). The starting strain for ethanol production, LY180, was more resistant to furfural than the starting strain for succinate production, XW055, (FIG. 9, FIG. 4C and FIG. 5C). However, the same combination of furfural-resistance traits was optimal for furfural tolerance with both strains. Genetic changes that increased furfural tolerance also increased resistance to hemicellulose hydrolysate, establishing the importance of furfural for toxicity and the generality of this approach. Although furfural is not the only inhibitor present in hydrolysate, enzymatic reduction of this compound should allow further studies to identify additional genes that confer resistance to remaining toxins. By developing biocatalysts that are resistant to furfural and other hemicellulose toxins, remaining toxins in hydrolysates can reduce the cost of fermentations by serving as a barrier that prevents the growth of undesirable contaminants.

Epistatic Interactions of Beneficial Traits for Furfural Tolerance

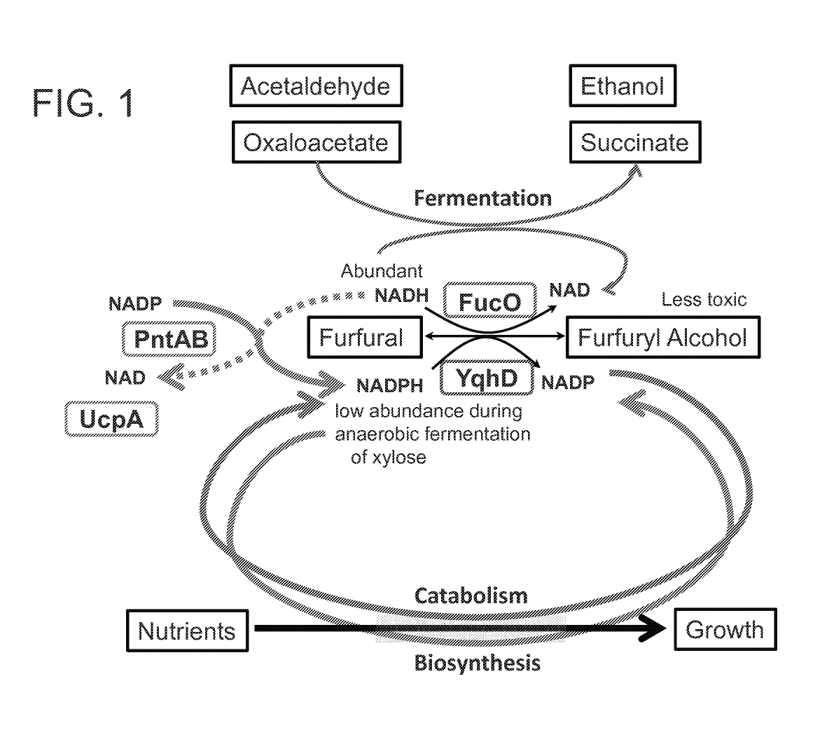

A general model is included to illustrate interactions among the 4 genetic traits for furfural tolerance (FIG. 1). Energy generation and growth require nutrients, intermediates from carbon catabolism, and balanced oxidation and regeneration of NADPH and NADH. YqhD has a low Kₘ for NADPH that competes effectively with biosynthesis, limiting growth by impeding NADPH-intensive processes such as sulfate assimilation (18). Increasing PntAB transhydrogenase partially restored this imbalance using NADH as a reductant (abundant during fermentation) (18). However, the combination of a yqhD deletion and increased expression of pntAB was more sensitive to furfural inhibition than either alone (FIG. 2B). NADPH-dependent furfural reductase YqhD may play a positive role for furfural tolerance in strains where pntAB expression has been increased. However, pyridine nucleotide transhydrogenase activity of PntAB couples proton translocation and makes the reduction of NADP by NADH a costly energy process (28). This increase in energy demand during expression of yqhD and pntAB could reduce fitness, despite potential benefits of reducing furfural to the less toxic alcohol. FucO can serve as a more effective furfural reductase because it utilizes NADH (abundant during fermentation) as the reductant, and does not compete for biosynthetic NADPH. Like pntAB, increased expression of ucpA in a yqhD deletion strain did not further increase furfural tolerance. This epistatic interaction suggests the UcpA-dependent furfural resistance may also involve NADPH availability (FIG. 2B).

Two furfural-resistant strains have been previously isolated and characterized, EMFR9 (selected for furfural tolerance; 19) and MM160 (selected for hydrolysate resistance; 17). Each contains a mutation that improves furfural tolerance by silencing YqhD using completely different mechanisms, IS10 disruption of adjacent yqhC (transcriptional activator for yqhD) and a nonsense mutation in yqhD, respectively (17, 20). Silencing genes such as yqhD can be caused by a myriad of genetic changes (29). An increase in fitness by gene silencing would be expected to emerge early in populations under growth-based selection. No mutations were found in these strains that increased expression of ucpA, pntAB, or fucO (13, 15, 18). Genetic solutions for gain of function mutants can be very limited and much less abundant (29, 30). Also, recovery of mutants with increased expression of ucpA and pntAB would be prevented by their negative interactions with yqhD silencing. Very high levels of fucO expression were needed that may require multiple mutations, dramatically limiting recovery without deliberate genetic constructions.

Succinate Fermentation from Lignocellulose Sugars

Succinic acid is currently produced from petroleum derived maleic anhydride and can serve as a starting material for synthesis of many commodity chemicals used in plastics and solvents (31). Genetically engineered strains of E. coli (32) and native succinate producers such as Actinobacillus succinogenes (33-35) and Anaerobiospirillum succiniciproducens (36) have been tested for lignocellulose conversion to succinate. However, fermentation using these strains required costly additional steps (33), nutrient supplementation (32-36), and mitigation of toxins in hydrolysates by overliming or treating with activated charcoal carbons (32, 35). Re-engineering derivatives of KJ122 using known combinations of furfural resistance traits resulted in strain XW136 that now ferments hemicellulose hydrolysates in mineral salts medium without costly detoxification steps (32 g/L succinic acid with a yield of 0.9 g/g sugars; FIG. 5C). The ability to use defined genetic traits for furfural tolerance to improve tolerance to inhibitors in hemicellulose hydrolysates should prove useful as a starting point for many new biocatalysts and products.

Materials and Methods

Strains and Growth Conditions.

Strains used are listed in Table 1. Ethanologenic E. coli LY180 (a derivative of E. coli W, ATCC 9637) and succinate-producing E. coli KJ122 (a derivative of E. coli C, ATCC 8739) were previously developed in our lab (19, 27). Strains XW092 (LY180. ΔyqhD), XW103 (LY180, adhE::pntAB), XW109(LY180. ΔyqhD adhE::pntAB), XW115 (LY180, ΔyqhD ackA::fucO-ucpA), XW116 (LY180, adhE::pntAB ackA::fucO-ucpA), XW129 (LY180, ΔyqhD ackA::P_{yadC′}fucO-ucpA) and XW131 (LY180, adhE::pntAB ackA::P_{yadC′}fucO-ucpA) were genetically engineered for furfural tolerance using LY180 as the parent strain. Strain KJ122 (succinate production from glucose) was serially transferred in pH-controlled fermenters (27) at 48 h intervals for approximately 40 generations to isolate a mutant with improved xylose fermentation (designated XW055). Strains XW120 (XW055, ΔyqhD ackA::P_{yadC′}fucO-ucpA) and XW136 (XW055, ΔyqhD ackA::P_{yadC′}fucO-ucpA adhE::fucO) were genetically engineered using XW055 as the parent strain. Cultures were grown in low salt xylose AM1 medium as previously described (37).

Genetic Methods.

Methods for seamless chromosomal deletion, gene replacement, or integration were previously described using Red recombinase technology (12, 27). Plasmids, primers, and construction details are listed in Table 1. Clone EZ® PCR Cloning Kit from GenScript (Piscataway, N.J.) was used for gene replacement on the plasmid. Constructions were made in Luria broth containing 20 g/L xylose, or 50 g/L arabinose (inducer for lambda Red recombinase; Gene Bridges GmbH, Heidelberg, Germany) or 100 g/L sucrose (for counter-selection of sacB). Antibiotics were added when required.

Identification of Promoter for fucO-ucpA Cassette.

A genome-wide promoter library with more than 10.000 clones was constructed in plasmid pLOI4870 (pACYC184 derivative) by ligating Sau3A1 fragments of E. coli genomic DNA into a unique BamHI site immediately upstream from a promoterless fucO-ucpA cassette (FIG. 6). The library was transformed into LY180 ΔyqhD cells with selection under argon for large colonies on AM1-xylose plates containing 12 mM furfural and 40 mg/L chloramphenicol. Of more than 10,000 transformants, 176 exhibited a large colony phenotype and were further compared using a BioScreen C growth curve analyzer (Piscataway, N.J.). The most effective clone was identified and designated plasmid pLOI5237 containing a 1,600 bp insert. Subcloning reduced the size of this promoter fragment to 600 bp (pLOI5259). This smaller fragment was identified by sequencing as part of the yadC coding region. The BamH1-furfural resistance cassette in pLOI4870 and pLOI5259 (includes upstream promoter fragment) were bordered by segments of ackA for chromosomal integration.

NADH-Dependent Furfural Reductase Assay and SDS-PAGE.

The preparation of cell crude lysates and furfural reductase assay were as previously described (17). Soluble protein lysates (15 μg protein) were also analyzed on 12% SDS PAGE gels (Bio-Rad, Hercules, Calif.).

Furfural Tolerance in Tube Cultures.

Furfural toxicity was measured in tube cultures (13 mm by 100 mm) as previously described for ethanol strains (17, 22). For succinate strains, tubes contained 4 ml of AM1 medium with 50 g/L xylose, 50 mM KHCO₃, and 100 mM MOPS as a buffer. Tubes were inoculated with starting cell density of 44 mg/L. Cell mass was measured at 550 nm after incubation for 48 h (37° C.).

Fermentation of Ethanol or Succinate.

Ethanol fermentations with xylose were carried out as previously described (17, 22), with and without furfural. For succinate production from xylose, seed pre-cultures of strains were grown in sealed culture tubes containing AM1 medium (20 g/L xylose, 50 mM KHCO₃ and 100 mM MOPS). After incubation for 16 h, pre-inocula were diluted into 500-ml fermentation vessels containing 300 ml AM1 media (100 g/L xylose, 1 mM betaine and 100 mM KHCO₃) at an initial density of 6.6 mg dry cell weight. After 24 h growth, these seed cultures were used to provide starting inocula for batch fermentations (AM1 medium, 100 g/L xylose and 100 mM KHCO₃). Fermentations were maintained at pH 7.0 by automatic addition of base containing additional CO₂ (2.4 M potassium carbonate in 1.2 M potassium hydroxide) as previously described (27). Quantitative analyses of sugars, ethanol, furfural, and succinate were as previously described (17, 27, 38).

Preparation and Fermentation of Hemicellulose Hydrolysates.

Hemicellulose hydrolysate was prepared as previously described (39, 40). Briefly, sugarcane bagasse (Florida Crystals Corporation, Okeelanta, Fla.) impregnated with phosphoric acid (0.5% of bagasse dry weight) was steam-treated for 5 min at 190° C. (39-41). Hemicellulose syrup (hydrolysate) was recovered using a screw press, discarding solids. After removal of fine particulates with a Whatman GF/D glass fiber filter, clarified hydrolysate was stored at 4° C. (pH 2.0). Hydrolysate was adjusted to pH 9.0 (5 M ammonium hydroxide) and stored for 16 h (22° C.) before use in fermentations, declining to pH 7.5. Batch fermentations (300 ml) were conducted in pH-controlled vessels containing 210 mL hemicelluloses hydrolysate supplemented with 0.5 mM sodium metabisulfite, components of AM1 medium (37), and inoculum. Potassium bicarbonate (100 mM) was included for succinate production. Final hydrolysate medium contained 36 g/L total sugar (primarily xylose), furfural 1.2 g/L, HMF 0.071 g/L, formic acid 1.1 g/L and acetic acid 3.2 g/L. Pre-cultures and seed cultures were prepared as described above. After 20 h incubation, seed cultures were used to provide a starting inoculum of 66 mg for hemicelluloses hydrolysate fermentations producing succinate or 13 mg for ethanol. Fermentations were maintained at pH 7.0 by the automatic addition of base (2.4 M potassium carbonate in 1.2 M potassium hydroxide for succinate or 2 N KOH for ethanol).

All patents, patent applications, provisional applications, and publications referred to or cited herein are incorporated by reference in their entirety, including all figures and tables, to the extent they are not inconsistent with the explicit teachings of this specification.

TABLE 1
Strains, plasmids and primers

| | | |
|---|---|---|
| Strains, | | |
| plasmids and | | Reference |
| primers | Relevant characteristics | of source |

Strains

| | | |
|---|---|---|
| LY 180 | ΔfrdBC: : (ZmfrgcelY_{Ec}), | (19) |
| | IdhA: : (ZmfrgcasAB_{Ko}), adhE: : (ZmfrgextZ_{Pp}FRT), ΔackA: : FRT, | |
| | rrlE: : (pdc adhA adhB FRT), ΔmgsA: : FRT | |
| XW092 | LY180 ΔyqhD | this study |
| XW103 | LY180 adhE: : pntAB | this study |
| XW109 | LY180 ΔyqhD adhE: : pntAB | this study |
| XW115 | LY180 ΔyqhD ackA: : fucO-ucpA | this study |
| XW116 | LY180 adhE: : pntAB ackA; : fucO-ucpA | this study |
| XW129 | LY180 ΔyqhD ackA: : P_{yadC′}: fucO-ucpA | this study |
| XW131 | LY180 adhE: : pntAB ackA: : P_{yadC′}: fucO-ucpA | this study |
| KJ122 | ΔadhE ΔldhA ΔfocA-pflB ΔtdcDE ΔmgsA ΔcitF ΔpoxB ΔaspC | (27) |
| | ΔsfcA ΔackA | |
| XW055 | KJ122 after serial transfer with xylose; succinate production | this study |
| | strain | |
| XW056 | XW055 ΔyqhD | this study |
| XW058 | XW055 adhE: : pntAB | this study |
| XW082 | XW055 ΔyqhD adhE: : pntAB | this study |
| XW120 | XW055 ΔyqhD ackA: : p_{yadC′}: fucO-ucpA | this study |
| XW135 | XW055 adhE: : pntAB ackA: : P_{yadC′}: fucO-ucpA | this study |
| XW136 | XW055 ΔyqhD ackA: : P_{yadC′}: fucO-ucpA adhE: : fucO | this study |

Plasmids
Characterization of epistatic interactions among furfural resistance traits

| | | |
|---|---|---|
| pCR2.1-TOPO | Bla, kan | Invitrogen |
| pTrc99a | pTrc bla oriR rrnB lacl^{q} | lab |
| | | collections |
| pTre fucO | fucO in pTrc99a | (17) |
| (pLOI4319) | | |
| pTrc ucpA | ucpA in pTrc99a | (22) |
| (pLOI4856) | | |
| pTrc fucO-ucpA | the intergenic region AATTGAAGAAGGAATAAGGT (SEQ ID | this study |
| (pLOI5229) | NO: 15) and E. coli ucpA ORF cloned after fucO ORF in | |
| | pLOI4319 | |

Promoter engineering and integration into ackA site

| | | |
|---|---|---|
| pACYC184 | cat tet p15A | NEB |
| pLOI4162 | bla, cat-sac cassette | (27) |
| pLOI4810 | PCR product of ackA region (ackA: : FRT and its adjacent regions) | this study |
| | of LY180 cloned into the pCR2.1-TOPO vector (Primers used: | |
| | ackA up and ackA down) | |
| pLOI4823 | cat-sacB cassette cloned into ackA region of pLOI4810 (primer | this study |
| | used: ackA 10 and ackA 20) | |
| pLOI4857 | E. coli ackA ORF and its adjacent regions (200 bp upstream and | this study |
| | downstream from coding region)(PCR) cloned into pACYC184 | |
| | by blunt ligation (Primers used: ackA up 200/ackA down 200; | |
| | pACYC-up/pACYC-down) | |
| pLOI4859 | ackA ORF in PLOI4857 was replaced by fucO-ucpA ORF (from | this study |
| | pLOI5229) by CloneEZ ® PCR Cloning Kit (primers used: ackA | |
| | 1/ackA 2; ackApAC up/ackApAC down) | |
| pLOI4869 | fUcO-ucpA ORF and ackA adjacent regions from pLOI4859 was | |
| | cloned into pACYC184. The tet ORF and its downstream | |
| | sequences (total 1.9 kb) were removed to reduce the size of the | |
| | plasmid smaller. (primers used: pACYC PacI/pACYC HindIlI: | |
| | HindIII ackA fucO/ackA fucO PacI) | |
| pLOI4870 | BamHI site and adhE RBS integrated before fucO-ucpA ORF in | this study |
| | pLOI4869 to provide ligation site to Sau3AI digested fragments | |
| | (primers used: fucO RBS and fucO BamHI) | |
| pLOI5237 | furfural resistant plasmid isolated by promoter screen | this study |
| PLOI5259 | PLOI5237 digested by BamHI and AatII and self-ligated. It | this study |
| | contains ackA: : P_{yadC′}: fucO-ucpA for chromosomal integration. | |

Plasmids used for strain constructions
Deletion of yqhD

| | | |
|---|---|---|
| PLOI5203 | E. coli yqhD and its adjacent regions (PCR) cloned into the | this study |
| | pCR2.1-TOPO vector | |
| pLOI5204 | cat-sacB cassette cloned into yqhD of PLOI5203 | this study |
| pLOI5205 | PacI digestion of pLOI5204, and self-ligated to delete yqhD ORF | this study |

Integration of adhE: : pntAB

| | | |
|---|---|---|
| PLOI5167 | E. coli adhE and its adjacent regions (PCR) from E. coli cloned | (17) |
| | into the pCR2;1-TOPO vector | |
| pLOI5168 | cat-sacB cassette cloned into adhE of pLOI5167 | (17) |
| pLOI5169 | PacI digestion of pLOI5168, and self-ligated to delete adhE ORF | (17) |
| pLOI5210 | Backbone of pACYC184 (PCR) bluntly ligated to adhE adjacent | this study |
| | regions (from pLOI5169)(primers used: pACYC-up/pACYC- | |
| | down; adhE up/adhE down) | |
| pLOI5214 | E. coli pntAB cloned into adhE adjacent regions in pLOI5210 to | this study |
| | accurately replace adhE ORF by CloneEZ ® PCR Cloning Kit | |
| | (primers used: adhE-pntAB ORF up/adhE-pntAB ORF down; | |
| | adhE-pntAB 1/adh E-pntAB 2) | |

Integration of adhE: : fucO

| | | |
|---|---|---|
| pLOI5209 | E. coli fucO ORF cloned into pLOI5167 to replace adhE ORF by | this study |
| | CloneEZ ® PCR Cloning Kit (primers used: adhE-fucO ORF | |
| | up/adhE-fucO ORF down; adhE-fucO 1/adhE-fucO 2) | |
| Primers | | this study |
| Deletion of yqhD | | this study |
| yqhD up | TATGATGCCAGGCTCGTACA (SEQ ID NO: 18) | this study |
| yqhD Down | GATCATGCCTTTCCATGCTT (SEQ ID NO: 19) | this study |
| yqhD 1 | GCTTTTTACGCCTCAAACTTTCGT (SEQ ID NO: 20) | this study |
| yqhD 2 | TACTTGCTCCCTTTGCTGG (SEQ ID NO: 21) | this study |
| Integration of adhE: : | | this study |
| pntAB | | |
| adhE up | CAATACGCCTTTTGACAGCA (SEQ ID NO: 22) | (17) |
| adhE down | GCCATCAATGGCAAAAAGTT (SEQ ID NO: 23) | (17) |
| adhE-pntAB ORF | TACTAAAAAAGTTTAACATTATCAGGAGAGCATTATGC | this study |
| up | GAATTGGCATACCAAGAG (SEQ ID NO: 24) | |
| adhE-pntAB ORF | TGCCAGACAGCGCTACTGATTACAGAGCTTTCAGGATT | this study |
| down | GCA (SEQ ID NO: 25) | |
| adhE-pntAB 1 | TGCAATCCTGAAAGCTCTGTAATCAGTAGCGCTGTCTG | this study |
| | GCA (SEQ ID NO: 26) | |
| adhE-pntAB 2 | CTCTTGGTATGCCAATTCGCATAATGCTCTCCTGATAAT | this study |
| | GTTAAACTTTTTTAGTA (SEQ ID NO: 27) | |
| pTre fucO-ucpA | | this study |
| construction | | |
| pTrefucO-UcpA | CTTGCCCGTGAGTTTACCCATACCTTATTCCTTCTTCAAT | this study |
| left | TTTACCAGGCGGTATGGTAAAGCT (SEQ ID NO: 28) | |
| pTreFucO-UcpA | CGGTTAGCGTCGGTATCTGAATGCGCTGATGTGATAAT | this study |
| right | GCCGGAT (SEQ ID NO: 29) | |
| pTreFucO-UcpA | AATTGAAGAAGGAATAAGGTATGGGTAAACTCACGGG | this study |
| ORFup | CAAG (SEQ ID NO: 30) | |
| pTreFucO-UcpA | ATCCGGCATTATCACATCAGCGCATTCAGATACCGACG | this study |
| ORF down | CTAACCG (SEQ ID NO: 31) | |
| Integration of ackA: : | | this study |
| fucO-ucpA | | |
| ackA 10 | GACTCTTCCGGCATAGTCTG (SEQ ID NO: 32) | this study |
| ackA 20 | GCATGAGCGTTGACGCAATC (SEQ ID NO: 33) | this study |
| ackA up | CTGGTTCTGAACTGCGGTAG (SEQ ID NO: 34) | this study |
| ackA down | CGCGATAACCAGTTCTTCGT (SEQ ID NO: 35) | this study |
| ackAup 200 | TTAGCAGCCTGAAGGCCTAA (SEQ ID NO: 36) | this study |
| ackAdown 200 | ACGACTTCAGCGTCTTTGGT (SEQ ID NO: 37) | this study |
| pACYC-up | CACCTCGCTAACGGATTCAC (SEQ ID NO: 38) | this study |
| pACYC-down | GGATGACGATGAGCGCATTG (SEQ ID NO: 39) | this study |
| ackA 1 | TTTCACACCGCCAGCTCAGC (SEQ ID NO: 40) | this study |
| ackA 2 | GGAAGTACCIATAATTGATACGTGGCTAAAAAAACGT | this study |
| | (SEQ ID NO: 41) | |
| ackApAC up | GTATCAATTATAGGTACTTCCATGATGGCTAACAGAAT | this study |
| | GATTCTG (SEQ ID NO: 42) | |
| ackApAC down | GCTGAGCTGGCGGTGTGAAATCAGATACCGACGCTAAC | this study |
| | CGTCTCC (SEQ ID NO: 43) | |
| pACYC PacI | GCATTTAATTAACCTGTGGAACACCTACATCT (SEQ ID | this study |
| | NO: 44) | |
| pACYC HindIII | AACCACTATGCCTACAG (SEQ ID NO: 45) | this study |
| HindIII ackA fucO | GCATAAGCTTTTAGCAGCCTGAAGGCCTAAGTAGTACA | this study |
| | TATTCAT (SEQ ID NO: 46) | |
| ackA fucO PacI | GCATTTAATTAAACGACTTCAGCGTCTTTGGTGTTAGCG | this study |
| | TG (SEQ ID NO: 47) | |
| fucO RBS | TATCAGGAGAGCATTATGATGGCTAACAGAATGATTCT | this study |
| | GAACGAAACG (SEQ ID NO: 48) | |
| fucO BamHI | GGATCCTGGCTAAAAAAACGTCAGGGAGCCATAGAGC | this study |
| | GTAGCGCATGATGA (SEQ ID NO: 49) | |

Integration of adhE: : fucO

| | | |
|---|---|---|
| adhE-fucO ORF | TACTAAAAAAGTTTAACATTATCAGGAGAGCATTATGA | this study |
| up | TGGCTAACAGAATGATTCTGAAC (SEQ ID NO: 50) | |
| adhE-fucO ORF | TGCCAGACAGCGCTACTGATTACCAGGCGGTATGGTAA | this study |
| down | AG (SEQ ID NO: 51) | |
| adhE-fucO 1 | CTTTACCATACCGCCTGGTAATCAGTAGCGCTGTCTGGC | this study |
| | A (SEQ ID NO: 52) | |
| adhE-fucO 2 | GTTCAGAATCATTCTGTTAGCCATCATAATGCTCTCCTG | this study |
| | ATAATGTTAAACTTTTTTAGTA (SEQ ID NO: 53) | |
| Sequencing of pLOI4870 | | this study |
| fucO ORF left | ACCAGCGTTTTATCGGTGAC (SEQ ID NO: 54) | this study |
| ackA up 200 | TTAGCAGCCTGAAGGCCTAA (SEQ ID NO: 55) | this study |

### REFERENCES

1. Carole T M, Pellegrino J, Paster M D (2004) Opportunities in the industrial biobased products industry. Appl Biochem Biotechnol 113-116:871-885.
2. Girio F M, et al. (2010) Hemicelluloses for fuel ethanol: A review. Bioresour Technol 101:4775-4800.
3. Saha B C (2003) Hemicellulose bioconversion. J Ind Microbiol Biotechnol 30:279-291.
4. Alvira P, Tomas-Pejo E, Ballesteros M. Negro M J (2010) Pretreatment technologies for an efficient bioethanol production process based on enzymatic hydrolysis: A review. Bioresour Technol 101:4851-4861.
5. Harris E E (1949) Wood Saccharification. Advances in Carbohydrate Chemistry 4:153-188.
6. Geddes C C, Nieves I U, Ingram L O (2011) Advances in ethanol production. Curr Opin Biotechnol 22:312-319.
7. Almeida J R, Bertilsson M, Gorwa-Grauslund M F, Gorsich S, Liden G (2009) Metabolic effects of furaldehydes and impacts on biotechnological processes. Appl Microbiol Biotechnol 82:625-638.
8. Mills T Y. Sandoval N R, Gill R T (2009) Cellulosic hydrolysate toxicity and tolerance mechanisms in Escherichia coli. Biotechnol Biofuels 2:26.
9. Martinez A, Rodriguez M E. York S W, Preston J F, Ingram L O (2000) Effects of Ca(OH)(2) treatments (“overliming”) on the composition and toxicity of bagasse hemicellulose hydrolysates. Biotechnol Bioeng 69:526-536.
10. Martinez A, et al. (2001) Detoxification of dilute acid hydrolysates of lignocellulose with lime. Biotechnol Prog 17:287-293.
11. Abbott D A, Zelle R M, Pronk J T, van Maris A J (2009) Metabolic engineering of Saccharomyces cerevisiae for production of carboxylic acids: current status and challenges. FEMS Yeast Res 9:1123-1136.
12. Jarboe L R. et al. (2010) Metabolic engineering for production of biorenewable fuels and chemicals: contributions of synthetic biology. J Biomed Biotechnol 2010:761042.
13. Zaldivar J, Martinez A, Ingram L O (1999) Effect of selected aldehydes on the growth and fermentation of ethanologenic Escherichia coli. Biotechnol Bioeng 65:24-33.
14. Taherzadeh M J, Gustafsson L, Niklasson C, Liden G (2000) Physiological effects of 5-hydroxymethylfurfural on Saccharomyces cerevisiae. Appl Microbiol Biotechnol 53:701-708.
15. Liu Z L, Blaschek H P (2010) Biomass conversion inhibitors and in situ detoxification. Biomass to biofiuels: strategies for global industries., ed Vertès A A, Blaschek H P, Yukawa H (John Wiley and Sons, West Sussex, United Kingdom), pp 233-259.
16. Liu Z L, Moon J, Andersh B J, Slininger P J, Weber S (2008) Multiple gene-mediated NAD(P)H-dependent aldehyde reduction is a mechanism of in situ detoxification of furfural and 5-hydroxymethylfurfural by Saccharomyces cerevisiae. Appl Microbiol Biotechnol 81:743-753.
17. Wang X, et al. (2011) Increased furfural tolerance due to overexpression of NADH-dependent oxidoreductase FucO in Escherichia coli strains engineered for the production of ethanol and lactate. Appl Environ Microbiol 77:5132-5140.
18. Miller E N, et al. (2009) Furfural inhibits growth by limiting sulfur assimilation in ethanologenic Escherichia coli strain LY180. Appl Environ MAicrobiol 75:6132-6141.
19. Miller E N, et al. (2009) Silencing of NADPH-dependent oxidoreductase genes (yqhD and dkgA) in furfural-resistant ethanologenic Escherichia coli. Appl Environ Microbiol 75:4315-4323.
20. Turner P C, et al. (2011) YqhC regulates transcription of the adjacent Escherichia coli genes yqhD and dkgA that are involved in furfural tolerance. J Ind Microbiol Biotechnol 38:431-439.
21. Miller E N, Turner P C, Jarboe L R, Ingram L O (2010) Genetic changes that increase 5-hydroxymethyl furfural resistance in ethanol-producing Escherichia coli LY180. Biotechnol Lett 32:661-667.
22. Wang X, Miller E N, Yomano L P, Shanmugam K T, Ingram L O (2012) Increased furan tolerance in Escherichia coli due to a cryptic ucpA gene. Appl Environ Microbiol 78:2452-2455.
23. Sawers R G (2006) Differential turnover of the multiple processed transcripts of the Escherichia coli focA-pflB operon. Microbiology 152:2197-2205.
24. Turner P C, et al. (2012) Optical mapping and sequencing of the Escherichia coli KO11 genome reveal extensive chromosomal rearrangements, and multiple tandem copies of the Zymomonas mobilis pdc and adhB genes. J Ind Microbiol Biotechnol 39:629-639.
25. Yomano L P, York S W, Zhou S, Shanmugam K T. Ingram L O (2008) Re-engineering Escherichia coli for ethanol production. Biotechnol Lett 30:2097-2103.
26. Jarboe L R, Grabar T B, Yomano L P, Shanmugan K T, Ingram L O (2007) Development of ethanologenic bacteria. Adv Biochem Eng Biotechnol 108:237-261.
27. Jantama K, et al. (2008) Eliminating side products and increasing succinate yields in engineered strains of Escherichia coli C. Biotechnol Bioeng 101:881-893.
28. Clarke D M, Bragg P D (1985) Purification and properties of reconstitutively active nicotinamide nucleotide transhydrogenase of Escherichia coli. Eur J Biochem 149:517-523.
29. Elena S F, Lenski R E (2003) Evolution experiments with microorganisms: the dynamics and genetic bases of adaptation. Nat Rev Genet 4:457-469.
30. Nam H, Conrad T M, Lewis N E (2011) The role of cellular objectives and selective pressures in metabolic pathway evolution. Curr Opin Biotechnol 22:595-600.
31. McKinlay J B, Vieille C, Zeikus J G (2007) Prospects for a bio-based succinate industry. Appl Microbiol Biotechnol 76:727-740.
32. Liu R. et al. (2012) Fermentation of xylose to succinate by enhancement of ATP supply in metabolically engineered Escherichia coli. Appl Microbiol Biotechnol.
33. Li Q, et al. (2010) Efficient conversion of crop stalk wastes into succinic acid production by Actinobacillus succinogenes. Bioresour Technol 101:3292-3294.
34. Zheng P, Dong J J, Sun Z H, Ni Y, Fang L (2009) Fermentative production of succinic acid from straw hydrolysate by Actinobacillus succinogenes. Bioresour Technol 100:2425-2429.
35. Borges E R, Pereira N, Jr. (2011) Succinic acid production from sugarcane bagasse hemicellulose hydrolysate by Actinobacillus succinogenes. J Ind Microbiol Biotechnol 38:1001-1011.
36. Lee P C. Lee S Y, Hong S H, Chang H N. Park S C (2003) Biological conversion of wood hydrolysate to succinic acid by Anaerobiospirillum succiniciproducens. Biotechnol Lett 25:111-114.
37. Martinez A, et al. (2007) Low salt medium for lactate and ethanol production by recombinant Escherichia coli B. Biotechnol Lett 29:397-404.
38. Martinez A. Rodriguez M E, York S W, Preston J F, Ingram L O (2000) Use of UV absorbance to monitor furans in dilute acid hydrolysates of biomass. Biotechnol Prog 16:637-641.
39. Geddes C C, et al. (2011) Simplified process for ethanol production from sugarcane bagasse using hydrolysate-resistant Escherichia coli strain MM160. Bioresour Technol. 102:2702-2711.
40. Geddes C C, et al. (2010) Optimizing the saccharification of sugar cane bagasse using dilute phosphoric acid followed by fungal cellulases. Bioresour Technol 101:1851-1857.
41. Varga E, Reczey K, Zacchi G (2004) Optimization of steam pretreatment of corn stover to enhance enzymatic digestibility. Appl Biochem Biotechnol 113-116:509-523.

1-58. (canceled)

59. An isolated ethanologenic or succinate producing bacterial strain comprising the following genetic modifications: plasmid expression of a fucO-ucpA construct or chromosomal integration of a fucO-ucpA construct into the gene encoding acetate kinase (ackA) of said bacterial strain in combination with the deletion of the gene encoding yqhD or chromosomal integration of a gene encoding pntAB behind the adhE promoter (adhE::pntAB).

60. The isolated ethanologenic or succinate producing bacterial strain according to claim 59, wherein the fucO-ucpA construct is operatively linked to a promoter sequence comprising
(SEQ ID NO: 1)
TTGAAAATGCAATGGTTGACTCAGGTAAAATGTATGGCTCCCATAAATTA
TTTAATACATCAGTTCCTGGTTTGTATTACACATT.

61. The isolated ethanologenic or succinate producing bacterial strain according to claim 60, wherein said promoter sequence comprises
(SEQ ID NO: 2)
ACCACGTGACTTATTAAAGATCTTTACTGCGGCTATACTCCTGCGACGCT
AATTGAGCAGCTTTTTGGTAAGATTGATCAAAAATGGAGAGAAACGGGGC
CGAATGGCGATGCTACTGTCATATTCAGATATGCAACAAGTACAAATAAT
TTAGTTTTCTACAAACCGACGCAGCTTGGACCTACAGGTGTAAAATTACA
GTGGAGTCAGTTAGATACCGCTTCTGGTGGTGGTTTTCTTTATTGCAACA
GAAGTGATAGCACAAGTGGTAGCGCAATGCGTATTGAAAATGCAATGGTT
GACTCAGGTAAAATGTATGGCTCCCATAAATTATTTAATACATCAGTTCC
TGGTTTGTATTACACATTATTAATTTCAAACATGTGGTCAGCTTACGGTA
CCGTAACTAACGTTAGTTCACCTGGGATATACATTGGTGACTCTGCAGAA
CAATATTTTTCGTGGTATAATCCAAGCGAAGACGTGTTATATTGGAGTTG
CAATAATGCGAATAGCACCCGTAAATACTGGGCTGTAGGTGGTATTTATC
AGACCCTTACAATTGAATTCTATACAGATACAAACTTTGATCCATATCAG
GAGAGCATT.

62. The isolated ethanologenic or succinate producing bacterial strain according to claim 60, wherein said promoter sequence comprises
(SEQ ID NO: 3)
ATCCACGTTTTGTATTAGCAAACGTCAAACTCTCATCGCTGACAGAACTCACC
GCAAAAGACCTTCTCGGTTAATGACCAGGGGCAGTGATCGTCTCATGGCCTTGCCAT
GGTGTTCTCTATGTTGCTGGCGGCGATTATCTGGAACCTGGGTACCTGGTACTTTGGT
TTACCTGCATCCAGCTCTCATACGCTGATTGGCGCGATCGCTTCTAAAGCAGCCCGC
ATGCGTTCCATCGTCGTTCCTGCGCCAGAAGCGCAAAATGATCGGCCAGCCCACCAG
GAAGCCCAGCGAGTAAATTAAGCCGTCATAGCCGGAGGTAAACACCAGCGCGGAGA
TCTCATTTTTAACCGCGTCAATCAGCATTGAAGAGTCCTGGCTTAAGTCATAGCCCG
GCGGATTAACCACCTGCATTTCCAGTTCAATACCGAGGGTAAAAGGTTCAGAAACAT
GAAAATCGGGTAATGGCATAGGTTTCTCTTAAGTTGGCGTTTTCTATTCAGTATAGA
AGTCGGAGCGGCTGGGCGAGATGCGGAAGTTCTGGAATGTTTCTTTTTTTGGTGATG
GTGACTGAAGCAATTTGGCTACTTTTGCAATGTGACAAGTTATGGCACGGCTGGCTG
GTGGCGAAGAATTTTGACGATTGAGGCATGCAGAAAAAAAACGGGTTCAGCTTTCA
GTTGATCCTCCCAGAACTTTGCTCTGGGGGGATACGGTCCCCGCTGTTCCCCGTCGCT
TAATCTGCATTATGCCGCGTAACTATGGCGCGGCGTTTAAGTTTCCTTGCCGATAGC
GGCGGCTGGCAGCGTTGGTTCTTTGCCGGTATTGCGATTGGTATTAGCGTGATCAAA
TTCCGCTGGCGGTTATCTCTGGCCCAACGTTTGCGAAAGAACTGGCGGCAGGTTTAC
CTACAGCTATTTCGCTGGCCTCGACCGATCAGGAATGCCCAGTGTTGTATTCAGACG
TCCACGTGACTTATTAAAGATCTTTACTGCGGCTATACTCCTGCGACGCTAATTGAG
CAGCTTTTTGGTAAGATTGATCAAAAATGGAGAGAAACGGGGCCGAATGGCGATGC
TACTGTCATATTCAGATATGCAACAAGTACAAATAATTTAGTTTTCTACAAACCGAC
GCAGCTTGGACCTACAGGTGTAAAATTACAGTGGAGTCAGTTAGATACCGCTTCTGG
TGGTGGTTTTCTTTATTGCAACAGAAGTGATAGCACAAGTGGTAGCGCAATGCGTAT
TGAAAATGCAATGGTTGACTCAGGTAAAATGTATGGCTCCCATAAATTATTTAA
TACATCAGTTCCTGGTTTGTATTACACATTATTAATTTCAAACATGTGGTCAGCTT
ACGGTACCGTAACTAACGTTAGTTCACCTGGGATATACATTGGTGACTCTGCAGAAC
AATATTTTTCGTGGTATAATCCAAGCGAAGACGTGTTATATTGGAGTTGCAATAATG
CGAATAGCACCCGTAAATACTGGGCTGTAGGTGGTATTTATCAGACCCTTACAATTG
AATTCTATACAGATACAAACTTTGATCCATATCAGGAGAGCATT.

63. The isolated ethanologenic or succinate producing bacterial strain according to claim 59, wherein said strain comprises the chromosomal integration of chromosomal integration of a fucO-ucpA construct comprising SEQ ID NO: 14 into the gene encoding acetate kinase (ackA) of said bacterial strain in combination with the deletion of the gene encoding yqhD.

64. The isolated ethanologenic or succinate producing bacterial strain according to claim 59, wherein said strain comprises the chromosomal integration of chromosomal integration of a fucO-ucpA construct into the gene encoding acetate kinase (ackA) of said bacterial strain in combination with chromosomal integration of a gene encoding pntAB behind the adhE promoter (adhE::pntAB; SEQ ID NO: 13).

65. The isolated ethanologenic or succinate producing bacterial strain according to claim 59, wherein said strain further comprises a plasmid comprising a gene encoding fucO operably linked to a promoter and/or a gene encoding fucO integrated into the chromosome of said bacterial strain and operably linked to a promoter native to said bacterial strain.

66. The isolated ethanologenic or succinate producing bacterial strain according to claim 65, wherein said strain comprises a gene encoding fucO integrated into the chromosome of said bacterial strain and operably linked to a promoter native to said bacterial strain.

67. The isolated ethanologenic or succinate producing bacterial strain according to claim 66, wherein said gene encoding fucO is integrated into the chromosome of said bacterial strain and operably linked to a promoter for alcohol/acetaldehyde dehydrogenase (adhE).

68. The isolated ethanologenic or succinate producing bacterial strain according to claim 66, wherein said gene encoding fucO is integrated into the chromosome of said bacterial strain, is operably linked to a promoter for alcohol/acetaldehyde dehydrogenase (adhE) and replaces the adhE gene in said bacterial strain.

69. An isolated bacterial, fungal or yeast cell comprising the following genetic modifications: plasmid expression of a fucO-ucpA construct or chromosomal integration of a fucO-ucpA construct into the gene encoding acetate kinase (ackA) of said bacterial, fungal or yeast cell in combination with the deletion of the gene encoding yqhD or chromosomal integration of a gene encoding pntAB behind the adhE promoter (adhE::pntAB).

70. The isolated bacterial, fungal or yeast cell according to claim 69, wherein the fucO-ucpA construct is operatively linked to a promoter sequence comprising
(SEQ ID NO: 1)
TTGAAAATGCAATGGTTGACTCAGGTAAAATGTATGGCTCCCATAAATTA
TTTAATACATCAGTTCCTGGTTTGTATTACACATT.

71. The isolated bacterial, fungal or yeast cell according to claim 70, wherein said promoter sequence comprises
(SEQ ID NO: 2)
ACCACGTGACTTATTAAAGATCTTTACTGCGGCTATACTCCTGCGACGCT
AATTGAGCAGCTTTTTGGTAAGATTGATCAAAAATGGAGAGAAACGGGGC
CGAATGGCGATGCTACTGTCATATTCAGATATGCAACAAGTACAAATAAT
TTAGTTTTCTACAAACCGACGCAGCTTGGACCTACAGGTGTAAAATTACA
GTGGAGTCAGTTAGATACCGCTTCTGGTGGTGGTTTTCTTTATTGCAACA
GAAGTGATAGCACAAGTGGTAGCGCAATGCGTATTGAAAATGCAATGGTT
GACTCAGGTAAAATGTATGGCTCCCATAAATTATTTAATACATCAGTTCC
TGGTTTGTATTACACATTATTAATTTCAAACATGTGGTCAGCTTACGGTA
CCGTAACTAACGTTAGTTCACCTGGGATATACATTGGTGACTCTGCAGAA
CAATATTTTTCGTGGTATAATCCAAGCGAAGACGTGTTATATTGGAGTTG
CAATAATGCGAATAGCACCCGTAAATACTGGGCTGTAGGTGGTATTTATC
AGACCCTTACAATTGAATTCTATACAGATACAAACTTTGATCCATATCAG
GAGAGCATT.

72. The isolated bacterial, fungal or yeast cell according to claim 70, wherein said promoter sequence comprises
(SEQ ID NO: 3)
ATCCACGTTTTGTATTAGCAAACGTCAAACTCTCATCGCTGACAGAACTCACC
GCAAAAGACCTTCTCGGTTAATGACCAGGGGCAGTGATCGTCTCATGGCCTTGCCAT
GGTGTTCTCTATGTTGCTGGCGGCGATTATCTGGAACCTGGGTACCTGGTACTTTGGT
TTACCTGCATCCAGCTCTCATACGCTGATTGGCGCGATCGCTTCTAAAGCAGCCCGC
ATGCGTTCCATCGTCGTTCCTGCGCCAGAAGCGCAAAATGATCGGCCAGCCCACCAG
GAAGCCCAGCGAGTAAATTAAGCCGTCATAGCCGGAGGTAAACACCAGCGCGGAGA
TCTCATTTTTAACCGCGTCAATCAGCATTGAAGAGTCCTGGCTTAAGTCATAGCCCG
GCGGATTAACCACCTGCATTTCCAGTTCAATACCGAGGGTAAAAGGTTCAGAAACAT
GAAAATCGGGTAATGGCATAGGTTTCTCTTAAGTTGGCGTTTTCTATTCAGTATAGA
AGTCGGAGCGGCTGGGCGAGATGCGGAAGTTCTGGAATGTTTCTTTTTTTGGTGATG
GTGACTGAAGCAATTTGGCTACTTTTGCAATGTGACAAGTTATGGCACGGCTGGCTG
GTGGCGAAGAATTTTGACGATTGAGGCATGCAGAAAAAAAACGGGTTCAGCTTTCA
GTTGATCCTCCCAGAACTTTGCTCTGGGGGGATACGGTCCCCGCTGTTCCCCGTCGCT
TAATCTGCATTATGCCGCGTAACTATGGCGCGGCGTTTAAGTTTCCTTGCCGATAGC
GGCGGCTGGCAGCGTTGGTTCTTTGCCGGTATTGCGATTGGTATTAGCGTGATCAAA
TTCCGCTGGCGGTTATCTCTGGCCCAACGTTTGCGAAAGAACTGGCGGCAGGTTTAC
CTACAGCTATTTCGCTGGCCTCGACCGATCAGGAATGCCCAGTGTTGTATTCAGACG
TCCACGTGACTTATTAAAGATCTTTACTGCGGCTATACTCCTGCGACGCTAATTGAG
CAGCTTTTTGGTAAGATTGATCAAAAATGGAGAGAAACGGGGCCGAATGGCGATGC
TACTGTCATATTCAGATATGCAACAAGTACAAATAATTTAGTTTTCTACAAACCGAC
GCAGCTTGGACCTACAGGTGTAAAATTACAGTGGAGTCAGTTAGATACCGCTTCTGG
TGGTGGTTTTCTTTATTGCAACAGAAGTGATAGCACAAGTGGTAGCGCAATGCGTAT
TGAAAATGCAATGGTTGACTCAGGTAAAATGTATGGCTCCCATAAATTATTTAA
TACATCAGTTCCTGGTTTGTATTACACATTATTAATTTCAAACATGTGGTCAGCTT
ACGGTACCGTAACTAACGTTAGTTCACCTGGGATATACATTGGTGACTCTGCAGAAC
AATATTTTTCGIGGTATAATCCAAGCGAAGACGTGTTATATTGGAGTTGCAATAATG
CGAATAGCACCCGTAAATACTGGGCTGTAGGTGGTATTTATCAGACCCTTACAATTG
AATTCTATACAGATACAAACTTTGATCCATATCAGGAGAGCATT.

73. The isolated bacterial, fungal or yeast cell according to claim 69, wherein said strain comprises the chromosomal integration of chromosomal integration of a fucO-ucpA construct comprising SEQ ID NO: 14 into the gene encoding acetate kinase (ackA) of said bacterial strain in combination with the deletion of the gene encoding yqhD.

74. The isolated bacterial, fungal or yeast cell according to claim 69, wherein said strain comprises the chromosomal integration of chromosomal integration of a fucO-ucpA construct into the gene encoding acetate kinase (ackA) of said bacterial strain in combination with chromosomal integration of a gene encoding pntAB behind the adhE promoter (adhE::pntAB; SEQ ID NO: 13).

75. The isolated bacterial, fungal or yeast cell according to claim 69, wherein said strain further comprises a plasmid comprising a gene encoding fucO operably linked to a promoter and/or a gene encoding fucO integrated into the chromosome of said bacterial strain and operably linked to a promoter native to said bacterial strain.

76. The isolated bacterial, fungal or yeast cell according to claim 75, wherein said strain comprises a gene encoding fucO integrated into the chromosome of said bacterial strain and operably linked to a promoter native to said bacterial strain.

77. The isolated bacterial, fungal or yeast cell according to claim 76, wherein said gene encoding fucO is integrated into the chromosome of said bacterial strain and operably linked to a promoter for alcohol/acetaldehyde dehydrogenase (adhE).

78. The isolated bacterial, fungal or yeast cell according to claim 76, wherein said gene encoding fucO is integrated into the chromosome of said bacterial strain, is operably linked to a promoter for alcohol/acetaldehyde dehydrogenase (adhE) and replaces the adhE gene in said bacterial strain.

79. A method of growing a bacterial cell comprising culturing a bacterial cell according to claim 59 under conditions that allow for the growth of said bacterial, fungal or yeast cell.

80. A method of increasing furfural and/or 5-hydroxymethylfurfural (5-HMF) resistance in a bacterial, fungal or yeast cell comprising introducing the following genetic modifications to said bacterial, fungal or yeast cell: plasmid expression of a fucO-ucpA construct or chromosomal integration of a fucO-ucpA construct into the gene encoding acetate kinase (ackA) of said bacterial, fungal or yeast cell in combination with the deletion of the gene encoding yqhD or chromosomal integration of a gene encoding pntAB behind the adhE promoter (adhE::pntAB).

81. An isolated bacterial, fungal or yeast cell comprising a fucO-ucpA construct, said fucO-ucpA construct encoding a lactaldehyde reductase and UcpA oxidoreductase activity.

82. The isolated bacterial, yeast or fungal cell according to claim 81, wherein said fucO-ucpA construct comprises SEQ ID NO: 14.
